(19) United States
(12) Patent Application Publication
(10) Pub. No.: US 2025/0160979 A1
(43) Pub. Date: May 22, 2025

(54) INTER-CONNECTIVITY OF DATA FLOWS BETWEEN INDEPENDENT SMART SYSTEMS

(71) Applicant: Cilag GmbH International, Zug (CH)

(72) Inventors: Frederick E. Shelton, IV, Hillsboro, OH (US); Jacqueline Corrigan Aronhalt, Loveland, OH (US); Kevin M. Fiebig, Cincinnati, OH (US); Taylor W. Aronhalt, Loveland, OH (US); Jason L. Harris, Lebanon, OH (US); Matthew David Cowperthwait, Cincinnati, OH (US); Nicholas James Ross, Franklin, OH (US); Shane R. Adams, Lebanon, OH (US)

(21) Appl. No.: 18/810,041

(22) Filed: Aug. 20, 2024

Related U.S. Application Data
(60) Provisional application No. 63/602,040, filed on Nov. 22, 2023.
(60) Provisional application No. 63/602,028, filed on Nov. 22, 2023.
(60) Provisional application No. 63/601,998, filed on Nov. 22, 2023.
(60) Provisional application No. 63/602,003, filed on Nov. 22, 2023.
(60) Provisional application No. 63/602,006, filed on Nov. 22, 2023.
(60) Provisional application No. 63/602,011, filed on Nov. 22, 2023.
(60) Provisional application No. 63/602,013, filed on Nov. 22, 2023.
(60) Provisional application No. 63/602,037, filed on Nov. 22, 2023.
(60) Provisional application No. 63/602,007, filed on Nov. 22, 2023.

(51) Int. Cl. A61B 34/37; G16H 40/67

(52) U.S. Cl. CPC A61B 34/37; G16H 40/67

(57) ABSTRACT

Systems, methods, and instrumentalities associated with adaptation of operation associated with monitoring of surgical devices. A first surgical device may monitor operation of a second surgical device. The monitoring of the second surgical device may affect the operation of the first surgical device (e.g., impact data generation). The first surgical device or the second surgical device may change its operating configuration, for example, based on the monitoring.

### CROSS-REFERENCE TO RELATED APPLICATIONS

This application claims the benefit of the following, the disclosures of which are incorporated herein by reference in its entirety:
Provisional U.S. Patent Application No. 63/602,040, filed Nov. 22, 2023;
Provisional U.S. Patent Application No. 63/602,028, filed Nov. 22, 2023;
Provisional U.S. Patent Application No. 63/601,998, filed Nov. 22, 2023,
Provisional U.S. Patent Application No. 63/602,003, filed Nov. 22, 2023,
Provisional U.S. Patent Application No. 63/602,006, filed Nov. 22, 2023,
Provisional U.S. Patent Application No. 63/602,011, filed Nov. 22, 2023,
Provisional U.S. Patent Application No. 63/602,013, filed Nov. 22, 2023,
Provisional U.S. Patent Application No. 63/602,037, filed Nov. 22, 2023, and
Provisional U.S. Patent Application No. 63/602,007, filed Nov. 22, 2023.

This application is related to the following, filed contemporaneously, the contents of each of which are incorporated by reference herein:
U.S. patent application Ser. No. 18/809,960, filed Aug. 20, 2024

### BACKGROUND

A surgical procedure may be performed within a surgical environment, such as an operating room. The surgical environment may include many interrelated systems and devices that communicate with each other to perform surgical procedures. Each surgical procedure may use tailored surgical environments with specific systems and/or devices.

Innovative medical technology may include interrelated systems that support and/or monitor each other throughout a surgical procedure. The interrelated systems may improve approaches to surgical procedures.

Throughout surgical procedures, the many interrelated systems may generate, send, and/or monitor various amounts of data and data types to each other to perform the procedure. Handling the vast amounts of data and data types presents many challenges.

### SUMMARY

Systems, methods, and instrumentalities are disclosed associated with adaptation of operation associated with monitoring of surgical devices. A first surgical device may monitor operation of a second surgical device. The monitoring of the second surgical device may affect the operation of the first surgical device (e.g., impact data generation). The first surgical device or the second surgical device may change its operating configuration, for example, based on the monitoring.

For example, a first surgical system may operate using a first operating configuration (e.g., first parameter). The first surgical system may determine first data, for example, using the first operating configuration. The first surgical system may determine that it is being monitored (e.g., by a second surgical system), for example, based on the first data. The first data may be impacted based on the monitoring. The first surgical system may determine an impact associated with the surgical system being monitored (e.g., impact on the first data). The first surgical system may determine an operating configuration to use based on the impact. The first surgical system may compare the impact with a threshold. For example, the first surgical system may determine to use (e.g., maintain) the first operating configuration if the impact is less than the threshold. For example, the first surgical system may determine to use (e.g., change to) a second operating configuration (e.g., second parameter) if the impact is greater than or equal to the threshold. The first surgical system may operate (e.g., generate second data) using the determined operating configuration.

For example, a first surgical system may monitor a second surgical system. The first surgical system may try to remain undetected by the second surgical system. The first surgical system may use a first operating configuration, for example, to monitor the second surgical system. The first surgical system may obtain first data (e.g., via the monitoring) associated with the second surgical system. The first surgical system may determine an impact associated with the second surgical system being monitored. The impact may be determined based on the first data. The first surgical system may determine an operating configuration to use based on the impact associated with the second surgical system being monitored. For example, the first surgical system may compare the impact with a threshold. For example, the first surgical system may determine to use (e.g., maintain) the first operating configuration if the impact is less than the threshold. For example, the first surgical system may determine to use (e.g., change to) a second operating configuration if the impact is greater than or equal to the threshold. The first surgical system may operate (e.g., monitor the first surgical system and/or obtain second data associated with the second surgical system) using the determined operating configuration.

### BRIEF DESCRIPTION OF THE DRAWINGS

FIG. 1 is a block diagram of a computer-implemented surgical system.

FIG. 2 shows an example surgical system in a surgical operating room.

FIG. 3 illustrates an example surgical hub paired with various systems.

FIG. 4 shows an example situationally aware surgical system.

FIG. 5 illustrates an example flow of interrelated surgical systems exchanging data within a surgical environment.

FIG. 6 illustrates an example flow of determining an operating configuration based on received data.

FIG. 7 illustrates an example flow of determining an operating configuration based on received data.

FIG. 8 illustrates an example flow of determining capability information associated with a surgical environment.

FIG. 9 illustrates an example of a smart battery charger sending usage information to a surgical hub.

FIG. 10 example illustrates an example of smart chargers for battery power stapling.

FIG. 11 illustrates an example flow of adapting operating configurations associated with data being monitored or received.

FIG. 12 illustrates an example flow of adapting operating configurations associated with data being monitored or received with respect to an impact on the data.

FIG. 13 illustrates an example flow of a surgical system changing its operating configuration to avoid being detected that it is monitoring a different surgical system.

FIG. 14A illustrates an example system adapting operating parameters based on a detection that the system is being monitored.

FIG. 14B illustrates an example of communication exchanged between cluster heads, the ground controller, and/or devices of a cluster.

FIG. 15 illustrates an example system adapting operating parameters to disturb a detected monitoring.

FIG. 16 illustrates an example encryption key passing to enable discreet monitoring of devices.

FIG. 17 illustrates an example surgical system that may include a surgical instrument.

### DETAILED DESCRIPTION

A more detailed understanding may be had from the following description, given by way of example in conjunction with the accompanying drawings.

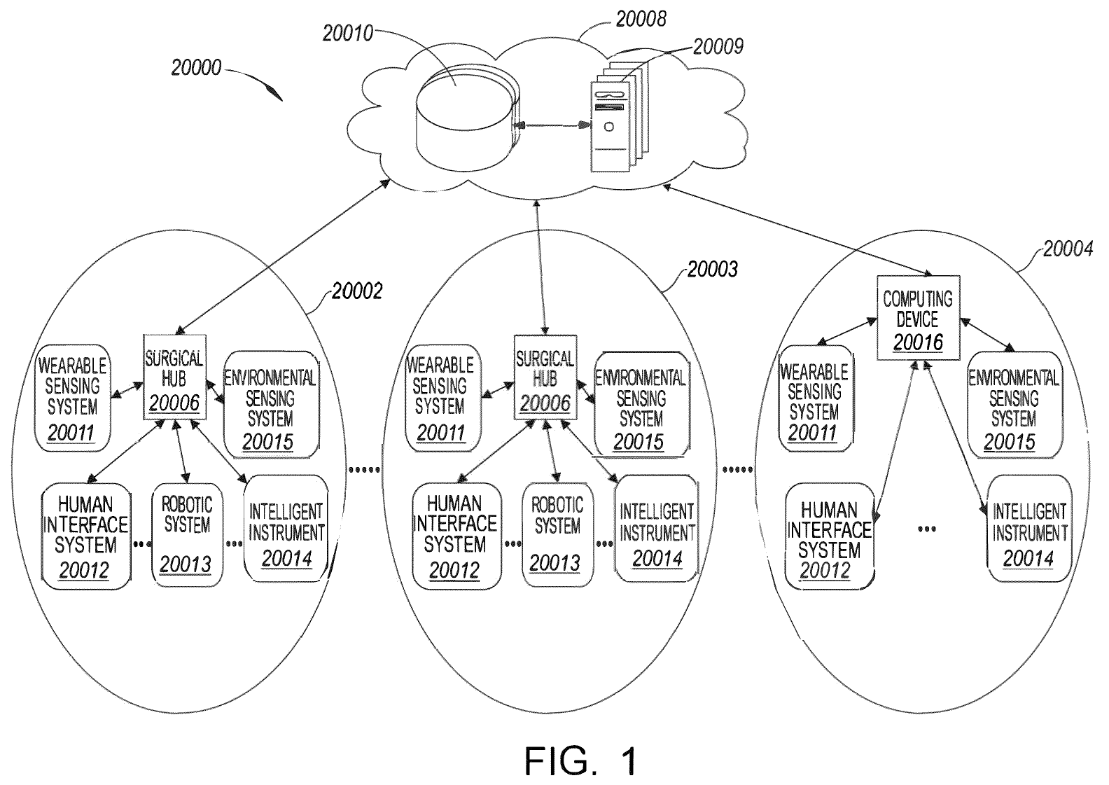

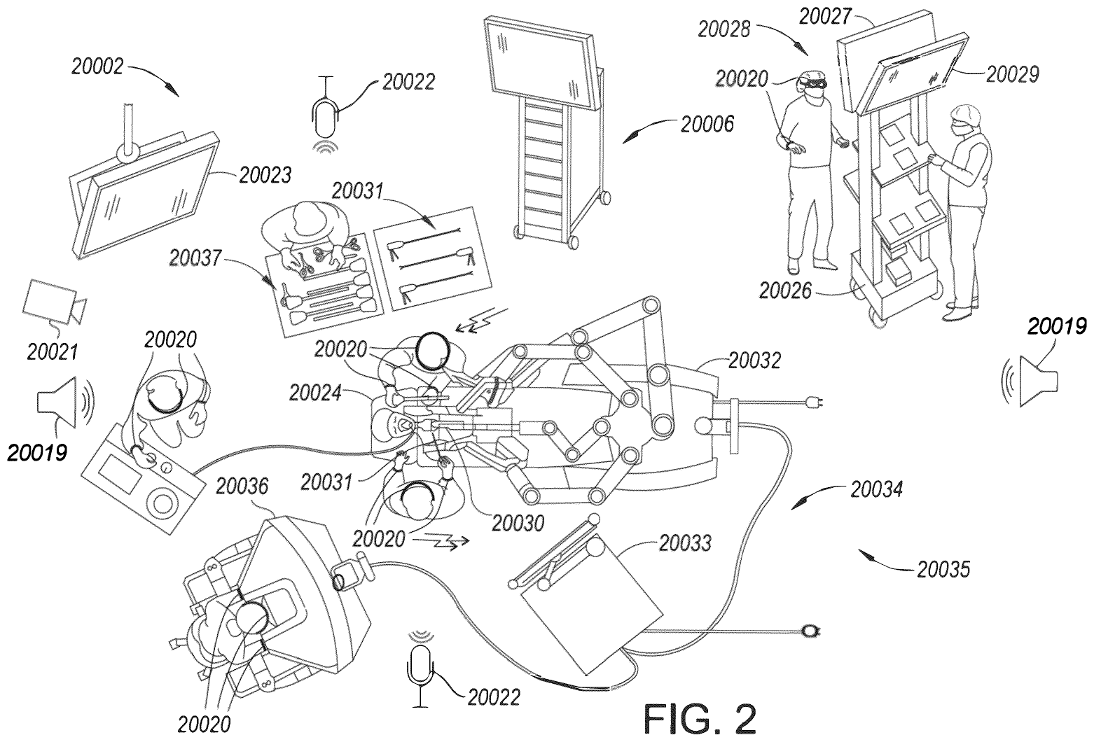

FIG. 1 shows an example computer-implemented surgical system 20000. The example surgical system 20000 may include one or more surgical systems (e.g., surgical sub-systems) 20002, 20003 and 20004. For example, surgical system 20002 may include a computer-implemented interactive surgical system. For example, surgical system 20002 may include a surgical hub 20006 and/or a computing device 20016 in communication with a cloud computing system 20008, for example, as described in FIG. 2. The cloud computing system 20008 may include at least one remote cloud server 20009 and at least one remote cloud storage unit 20010. Example surgical systems 20002, 20003, or 20004 may include one or more wearable sensing systems 20011, one or more environmental sensing systems 20015, one or more robotic systems 20013, one or more intelligent instruments 20014, one or more human interface systems 20012, etc. The human interface system is also referred herein as the human interface device. The wearable sensing system 20011 may include one or more health care professional (HCP) sensing systems, and/or one or more patient sensing systems. The environmental sensing system 20015 may include one or more devices, for example, used for measuring one or more environmental attributes, for example, as further described in FIG. 2. The robotic system 20013 may include a plurality of devices used for performing a surgical procedure, for example, as further described in FIG. 2.

The surgical system 20002 may be in communication with a remote server 20009 that may be part of a cloud computing system 20008. In an example, the surgical system 20002 may be in communication with a remote server 20009 via an internet service provider's cable/FIOS networking node. In an example, a patient sensing system may be in direct communication with a remote server 20009. The surgical system 20002 (and/or various sub-systems, smart surgical instruments, robots, sensing systems, and other computerized devices described herein) may collect data in real-time and transfer the data to cloud computers for data processing and manipulation. It will be appreciated that cloud computing may rely on sharing computing resources rather than having local servers or personal devices to handle software applications.

The surgical system 20002 and/or a component therein may communicate with the remote servers 20009 via a cellular transmission/reception point (TRP) or a base station using one or more of the following cellular protocols: GSM/GPRS/EDGE (2G), UMTS/HSPA (3G), long term evolution (LTE) or 4G, LTE-Advanced (LTE-A), new radio (NR) or 5G, and/or other wired or wireless communication protocols. Various examples of cloud-based analytics that are performed by the cloud computing system 20008, and are suitable for use with the present disclosure, are described in U.S. Patent Application Publication No. US 2019-0206569 A1 (U.S. patent application Ser. No. 16/209,403), titled METHOD OF CLOUD BASED DATA ANALYTICS FOR USE WITH THE HUB, filed Dec. 4, 2018, the disclosure of which is herein incorporated by reference in its entirety.

The surgical hub 20006 may have cooperative interactions with one of more means of displaying the image from the laparoscopic scope and information from one or more other smart devices and one or more sensing systems 20011. The surgical hub 20006 may interact with one or more sensing systems 20011, one or more smart devices, and multiple displays. The surgical hub 20006 may be configured to gather measurement data from the sensing system(s) and send notifications or control messages to the one or more sensing systems 20011. The surgical hub 20006 may send and/or receive information including notification information to and/or from the human interface system 20012. The human interface system 20012 may include one or more human interface devices (HIDs). The surgical hub 20006 may send and/or receive notification information or control information to audio, display and/or control information to various devices that are in communication with the surgical hub.

For example, the sensing systems may include the wearable sensing system 20011 (which may include one or more HCP sensing systems and/or one or more patient sensing systems) and/or the environmental sensing system 20015 shown in FIG. 1. The sensing system(s) may measure data relating to various biomarkers. The sensing system(s) may measure the biomarkers using one or more sensors, for example, photosensors (e.g., photodiodes, photoresistors), mechanical sensors (e.g., motion sensors), acoustic sensors, electrical sensors, electrochemical sensors, thermoelectric sensors, infrared sensors, etc. The sensor(s) may measure the biomarkers as described herein using one of more of the following sensing technologies: photoplethysmography, electrocardiography, electroencephalography, colorimetry, impedimentary, potentiometry, amperometry, etc.

The biomarkers measured by the sensing systems may include, but are not limited to, sleep, core body temperature, maximal oxygen consumption, physical activity, alcohol consumption, respiration rate, oxygen saturation, blood pressure, blood sugar, heart rate variability, blood potential of hydrogen, hydration state, heart rate, skin conductance, peripheral temperature, tissue perfusion pressure, coughing and sneezing, gastrointestinal motility, gastrointestinal tract imaging, respiratory tract bacteria, edema, mental aspects, sweat, circulating tumor cells, autonomic tone, circadian rhythm, and/or menstrual cycle.

The biomarkers may relate to physiologic systems, which may include, but are not limited to, behavior and psychology, cardiovascular system, renal system, skin system, nervous system, gastrointestinal system, respiratory system, endocrine system, immune system, tumor, musculoskeletal system, and/or reproductive system. Information from the biomarkers may be determined and/or used by the computer-implemented patient and the surgical system 20000, for example. The information from the biomarkers may be determined and/or used by the computer-implemented patient and the surgical system 20000 to improve said systems and/or to improve patient outcomes, for example.

The sensing systems may send data to the surgical hub 20006. The sensing systems may use one or more of the following RF protocols for communicating with the surgical hub 20006: Bluetooth, Bluetooth Low-Energy (BLE), Bluetooth Smart, Zigbee, Z-wave, IPv6 Low-power wireless Personal Area Network (6LoWPAN), Wi-Fi.

The sensing systems, biomarkers, and physiological systems are described in more detail in U.S. application Ser. No. 17/156,287 (attorney docket number END9290USNP1), titled METHOD OF ADJUSTING A SURGICAL PARAMETER BASED ON BIOMARKER MEASUREMENTS, filed Jan. 22, 2021, the disclosure of which is herein incorporated by reference in its entirety.

The sensing systems described herein may be employed to assess physiological conditions of a surgeon operating on a patient or a patient being prepared for a surgical procedure or a patient recovering after a surgical procedure. The cloud-based computing system 20008 may be used to monitor biomarkers associated with a surgeon or a patient in real-time and to generate surgical plans based at least on measurement data gathered prior to a surgical procedure, provide control signals to the surgical instruments during a surgical procedure, and notify a patient of a complication during post-surgical period.

The cloud-based computing system 20008 may be used to analyze surgical data. Surgical data may be obtained via one or more intelligent instrument(s) 20014, wearable sensing system(s) 20011, environmental sensing system(s) 20015, robotic system(s) 20013 and/or the like in the surgical system 20002. Surgical data may include, tissue states to assess leaks or perfusion of sealed tissue after a tissue sealing and cutting procedure pathology data, including images of samples of body tissue, anatomical structures of the body using a variety of sensors integrated with imaging devices and techniques such as overlaying images captured by multiple imaging devices, image data, and/or the like. The surgical data may be analyzed to improve surgical procedure outcomes by determining if further treatment, such as the application of endoscopic intervention, emerging technologies, a targeted radiation, targeted intervention, and precise robotics to tissue-specific sites and conditions. Such data analysis may employ outcome analytics processing and using standardized approaches may provide beneficial feedback to either confirm surgical treatments and the behavior of the surgeon or suggest modifications to surgical treatments and the behavior of the surgeon.

FIG. 2 shows an example surgical system 20002 in a surgical operating room. As illustrated in FIG. 2, a patient is being operated on by one or more health care professionals (HCPs). The HCPs are being monitored by one or more HCP sensing systems 20020 worn by the HCPs. The HCPs and the environment surrounding the HCPs may also be monitored by one or more environmental sensing systems including, for example, a set of cameras 20021, a set of microphones 20022, and other sensors that may be deployed in the operating room. The HCP sensing systems 20020 and the environmental sensing systems may be in communication with a surgical hub 20006, which in turn may be in communication with one or more cloud servers 20009 of the cloud computing system 20008, as shown in FIG. 1. The environmental sensing systems may be used for measuring one or more environmental attributes, for example, HCP position in the surgical theater, HCP movements, ambient noise in the surgical theater, temperature/humidity in the surgical theater, etc.

As illustrated in FIG. 2, a primary display 20023 and one or more audio output devices (e.g., speakers 20019) are positioned in the sterile field to be visible to an operator at the operating table 20024. In addition, a visualization/notification tower 20026 is positioned outside the sterile field. The visualization/notification tower 20026 may include a first non-sterile human interactive device (HID) 20027 and a second non-sterile HID 20029, which may face away from each other. The HID may be a display or a display with a touchscreen allowing a human to interface directly with the HID. A human interface system, guided by the surgical hub 20006, may be configured to utilize the HIDs 20027, 20029, and 20023 to coordinate information flow to operators inside and outside the sterile field. In an example, the surgical hub 20006 may cause an HID (e.g., the primary HID 20023) to display a notification and/or information about the patient and/or a surgical procedure step. In an example, the surgical hub 20006 may prompt for and/or receive input from personnel in the sterile field or in the non-sterile area. In an example, the surgical hub 20006 may cause an HID to display a snapshot of a surgical site, as recorded by an imaging device 20030, on a non-sterile HID 20027 or 20029, while maintaining a live feed of the surgical site on the primary HID 20023. The snapshot on the non-sterile display 20027 or 20029 can permit a non-sterile operator to perform a diagnostic step relevant to the surgical procedure, for example.

The surgical hub 20006 may be configured to route a diagnostic input or feedback entered by a non-sterile operator at the visualization tower 20026 to the primary display 20023 within the sterile field, where it can be viewed by a sterile operator at the operating table. In an example, the input can be in the form of a modification to the snapshot displayed on the non-sterile display 20027 or 20029, which can be routed to the primary display 20023 by the surgical hub 20006.

Referring to FIG. 2, a surgical instrument 20031 is being used in the surgical procedure as part of the surgical system 20002. The hub 20006 may be configured to coordinate information flow to a display of the surgical instrument(s) 20031. For example, in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. A diagnostic input or feedback entered by a non-sterile operator at the visualization tower 20026 can be routed by the hub 20006 to the surgical instrument display within the sterile field, where it can be viewed by the operator of the surgical instrument 20031. Example surgical instruments that are suitable for use with the surgical system 20002 are described under the heading “Surgical Instrument Hardware” and in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety, for example.

As shown in FIG. 2, the surgical system 20002 can be used to perform a surgical procedure on a patient who is lying down on an operating table 20024 in a surgical operating room 20035. A robotic system 20034 may be used in the surgical procedure as a part of the surgical system 20002. The robotic system 20034 may include a surgeon's console 20036, a patient side cart 20032 (surgical robot), and a surgical robotic hub 20033. The patient side cart 20032 can manipulate at least one removably coupled surgical tool 20037 through a minimally invasive incision in the body of the patient while the surgeon views the surgical site through the surgeon's console 20036. An image of the surgical site can be obtained by a medical imaging device 20030, which can be manipulated by the patient side cart 20032 to orient the imaging device 20030. The robotic hub 20033 can be used to process the images of the surgical site for subsequent display to the surgeon through the surgeon's console 20036.

Other types of robotic systems can be readily adapted for use with the surgical system 20002. Various examples of robotic systems and surgical tools that are suitable for use with the present disclosure are described herein, as well as in U.S. Patent Application Publication No. US 2019-0201137 A1 (U.S. patent application Ser. No. 16/209,407), titled METHOD OF ROBOTIC HUB COMMUNICATION, DETECTION, AND CONTROL, filed Dec. 4, 2018, the disclosure of which is herein incorporated by reference in its entirety.

In various aspects, the imaging device 20030 may include at least one image sensor and one or more optical components. Suitable image sensors may include, but are not limited to, Charge-Coupled Device (CCD) sensors and Complementary Metal-Oxide Semiconductor (CMOS) sensors.

The optical components of the imaging device 20030 may include one or more illumination sources and/or one or more lenses. The one or more illumination sources may be directed to illuminate portions of the surgical field. The one or more image sensors may receive light reflected or refracted from the surgical field, including light reflected or refracted from tissue and/or surgical instruments.

The illumination source(s) may be configured to radiate electromagnetic energy in the visible spectrum as well as the invisible spectrum. The visible spectrum, sometimes referred to as the optical spectrum or luminous spectrum, is the portion of the electromagnetic spectrum that is visible to (e.g., can be detected by) the human eye and may be referred to as visible light or simply light. A typical human eye will respond to wavelengths in air that range from about 380 nm to about 750 nm.

The invisible spectrum (e.g., the non-luminous spectrum) is the portion of the electromagnetic spectrum that lies below and above the visible spectrum (i.e., wavelengths below about 380 nm and above about 750 nm). The invisible spectrum is not detectable by the human eye. Wavelengths greater than about 750 nm are longer than the red visible spectrum, and they become invisible infrared (IR), microwave, and radio electromagnetic radiation. Wavelengths less than about 380 nm are shorter than the violet spectrum, and they become invisible ultraviolet, x-ray, and gamma ray electromagnetic radiation.

In various aspects, the imaging device 20030 is configured for use in a minimally invasive procedure. Examples of imaging devices suitable for use with the present disclosure include, but are not limited to, an arthroscope, angioscope, bronchoscope, choledochoscope, colonoscope, cytoscope, duodenoscope, enteroscope, esophagogastro-duodenoscope (gastroscope), endoscope, laryngoscope, nasopharyngo-neproscope, sigmoidoscope, thoracoscope, and ureteroscope.

The imaging device may employ multi-spectrum monitoring to discriminate topography and underlying structures. A multi-spectral image is one that captures image data within specific wavelength ranges across the electromagnetic spectrum. The wavelengths may be separated by filters or by the use of instruments that are sensitive to particular wavelengths, including light from frequencies beyond the visible light range, e.g., IR and ultraviolet. Spectral imaging can allow extraction of additional information that the human eye fails to capture with its receptors for red, green, and blue. The use of multi-spectral imaging is described in greater detail under the heading “Advanced Imaging Acquisition Module” in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. Multi-spectrum monitoring can be a useful tool in relocating a surgical field after a surgical task is completed to perform one or more of the previously described tests on the treated tissue. It is axiomatic that strict sterilization of the operating room and surgical equipment is required during any surgery. The strict hygiene and sterilization conditions required in a “surgical theater,” e.g., an operating or treatment room, necessitate the highest possible sterility of all medical devices and equipment. Part of that sterilization process is the need to sterilize anything that comes in contact with the patient or penetrates the sterile field, including the imaging device 20030 and its attachments and components. It will be appreciated that the sterile field may be considered a specified area, such as within a tray or on a sterile towel, that is considered free of microorganisms, or the sterile field may be considered an area, immediately around a patient, who has been prepared for a surgical procedure. The sterile field may include the scrubbed team members, who are properly attired, and all furniture and fixtures in the area.

Wearable sensing system 20011 illustrated in FIG. 1 may include one or more HCP sensing systems 20020 as shown in FIG. 2. The HCP sensing systems 20020 may include sensing systems to monitor and detect a set of physical states and/or a set of physiological states of a healthcare personnel (HCP). An HCP may be a surgeon or one or more healthcare personnel assisting the surgeon or other healthcare service providers in general. In an example, an HCP sensing system 20020 may measure a set of biomarkers to monitor the heart rate of an HCP. In an example, an HCP sensing system 20020 worn on a surgeon's wrist (e.g., a watch or a wristband) may use an accelerometer to detect hand motion and/or shakes and determine the magnitude and frequency of tremors. The sensing system 20020 may send the measurement data associated with the set of biomarkers and the data associated with a physical state of the surgeon to the surgical hub 20006 for further processing.

The environmental sensing system(s) 20015 shown in FIG. 1 may send environmental information to the surgical hub 20006. For example, the environmental sensing system(s) 20015 may include a camera 20021 for detecting hand/body position of an HCP. The environmental sensing system(s) 20015 may include microphones 20022 for measuring the ambient noise in the surgical theater. Other environmental sensing system(s) 20015 may include devices, for example, a thermometer to measure temperature and a hygrometer to measure humidity of the surroundings in the surgical theater, etc. The surgeon biomarkers may include one or more of the following: stress, heart rate, etc. The environmental measurements from the surgical theater may include ambient noise level associated with the surgeon or the patient, surgeon and/or staff movements, surgeon and/or staff attention level, etc. The surgical hub 20006, alone or in communication with the cloud computing system, may use the surgeon biomarker measurement data and/or environmental sensing information to modify the control algorithms of hand-held instruments or the averaging delay of a robotic interface, for example, to minimize tremors.

The surgical hub 20006 may use the surgeon biomarker measurement data associated with an HCP to adaptively control one or more surgical instruments 20031. For example, the surgical hub 20006 may send a control program to a surgical instrument 20031 to control its actuators to limit or compensate for fatigue and use of fine motor skills. The surgical hub 20006 may send the control program based on situational awareness and/or the context on importance or criticality of a task. The control program may instruct the instrument to alter operation to provide more control when control is needed.

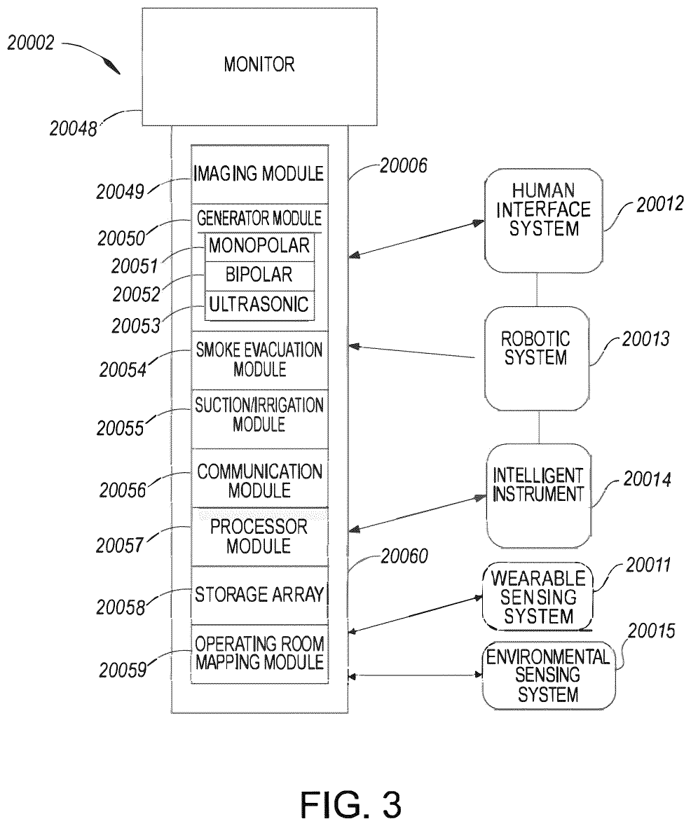

FIG. 3 shows an example surgical system 20002 with a surgical hub 20006. The surgical hub 20006 may be paired with, via a modular control, a wearable sensing system 20011, an environmental sensing system 20015, a human interface system 20012, a robotic system 20013, and an intelligent instrument 20014. The hub 20006 includes a display 20048, an imaging module 20049, a generator module 20050 (e.g., an energy generator), a communication module 20056, a processor module 20057, a storage array 20058, and an operating-room mapping module 20059. In certain aspects, as illustrated in FIG. 3, the hub 20006 further includes a smoke evacuation module 20054 and/or a suction/irrigation module 20055. The various modules and systems may be connected to the modular control either directly via a router or via the communication module 20056. The operating theater devices may be coupled to cloud computing resources and data storage via the modular control. The human interface system 20012 may include a display sub-system and a notification sub-system.

The modular control may be coupled to non-contact sensor module. The non-contact sensor module may measure the dimensions of the operating theater and generate a map of the surgical theater using, ultrasonic, laser-type, and/or the like, non-contact measurement devices. Other distance sensors can be employed to determine the bounds of an operating room. An ultrasound-based non-contact sensor module may scan the operating theater by transmitting a burst of ultrasound and receiving the echo when it bounces off the perimeter walls of an operating theater as described under the heading “Surgical Hub Spatial Awareness Within an Operating Room” in U.S. Provisional Patent Application Ser. No. 62/611,341, titled INTERACTIVE SURGICAL PLATFORM, filed Dec. 28, 2017, which is herein incorporated by reference in its entirety. The sensor module may be configured to determine the size of the operating theater and to adjust Bluetooth-pairing distance limits. A laser-based non-contact sensor module may scan the operating theater by transmitting laser light pulses, receiving laser light pulses that bounce off the perimeter walls of the operating theater, and comparing the phase of the transmitted pulse to the received pulse to determine the size of the operating theater and to adjust Bluetooth pairing distance limits, for example.

During a surgical procedure, energy application to tissue, for sealing and/or cutting, may be associated with smoke evacuation, suction of excess fluid, and/or irrigation of the tissue. Fluid, power, and/or data lines from different sources may be entangled during the surgical procedure. Valuable time can be lost addressing this issue during a surgical procedure. Detangling the lines may necessitate disconnecting the lines from their respective modules, which may require resetting the modules. The hub modular enclosure 20060 may offer a unified environment for managing the power, data, and fluid lines, which reduces the frequency of entanglement between such lines.

Energy may be applied to tissue at a surgical site. The surgical hub 20006 may include a hub enclosure 20060 and a combo generator module slidably receivable in a docking station of the hub enclosure 20060. The docking station may include data and power contacts. The combo generator module may include two or more of: an ultrasonic energy generator component, a bipolar RF energy generator component, or a monopolar RF energy generator component that are housed in a single unit. The combo generator module may include a smoke evacuation component, at least one energy delivery cable for connecting the combo generator module to a surgical instrument, at least one smoke evacuation component configured to evacuate smoke, fluid, and/or particulates generated by the application of therapeutic energy to the tissue, and a fluid line extending from the remote surgical site to the smoke evacuation component. The fluid line may be a first fluid line, and a second fluid line may extend from the remote surgical site to a suction and irrigation module 20055 slidably received in the hub enclosure 20060. The hub enclosure 20060 may include a fluid interface.

The combo generator module may generate multiple energy types for application to the tissue. One energy type may be more beneficial for cutting the tissue, while another different energy type may be more beneficial for sealing the tissue. For example, a bipolar generator can be used to seal the tissue while an ultrasonic generator can be used to cut the sealed tissue. Aspects of the present disclosure present a solution where a hub modular enclosure 20060 is configured to accommodate different generators and facilitate an interactive communication therebetween. The hub modular enclosure 20060 may enable the quick removal and/or replacement of various modules.

The modular surgical enclosure may include a first energy-generator module, configured to generate a first energy for application to the tissue, and a first docking station comprising a first docking port that includes first data and power contacts, wherein the first energy-generator module is slidably movable into an electrical engagement with the power and data contacts and wherein the first energy-generator module is slidably movable out of the electrical engagement with the first power and data contacts. The modular surgical enclosure may include a second energy-generator module configured to generate a second energy, different than the first energy, for application to the tissue, and a second docking station comprising a second docking port that includes second data and power contacts, wherein the second energy generator module is slidably movable into an electrical engagement with the power and data contacts, and wherein the second energy-generator module is slidably movable out of the electrical engagement with the second power and data contacts. In addition, the modular surgical enclosure also includes a communication bus between the first docking port and the second docking port, configured to facilitate communication between the first energy-generator module and the second energy-generator module.

Referring to FIG. 3, the hub modular enclosure 20060 may allow the modular integration of a generator module 20050, a smoke evacuation module 20054, and a suction/irrigation module 20055. The hub modular enclosure 20060 may facilitate interactive communication between the modules 20059, 20054, and 20055. The generator module 20050 can be with integrated monopolar, bipolar, and ultrasonic components supported in a single housing unit slidably insertable into the hub modular enclosure 20060. The generator module 20050 may connect to a monopolar device 20051, a bipolar device 20052, and an ultrasonic device 20053. The generator module 20050 may include a series of monopolar, bipolar, and/or ultrasonic generator modules that interact through the hub modular enclosure 20060. The hub modular enclosure 20060 may facilitate the insertion of multiple generators and interactive communication between the generators docked into the hub modular enclosure 20060 so that the generators would act as a single generator.

A surgical data network having a set of communication hubs may connect the sensing system(s), the modular devices located in one or more operating theaters of a healthcare facility, a patient recovery room, or a room in a healthcare facility specially equipped for surgical operations, to the cloud computing system 20008.

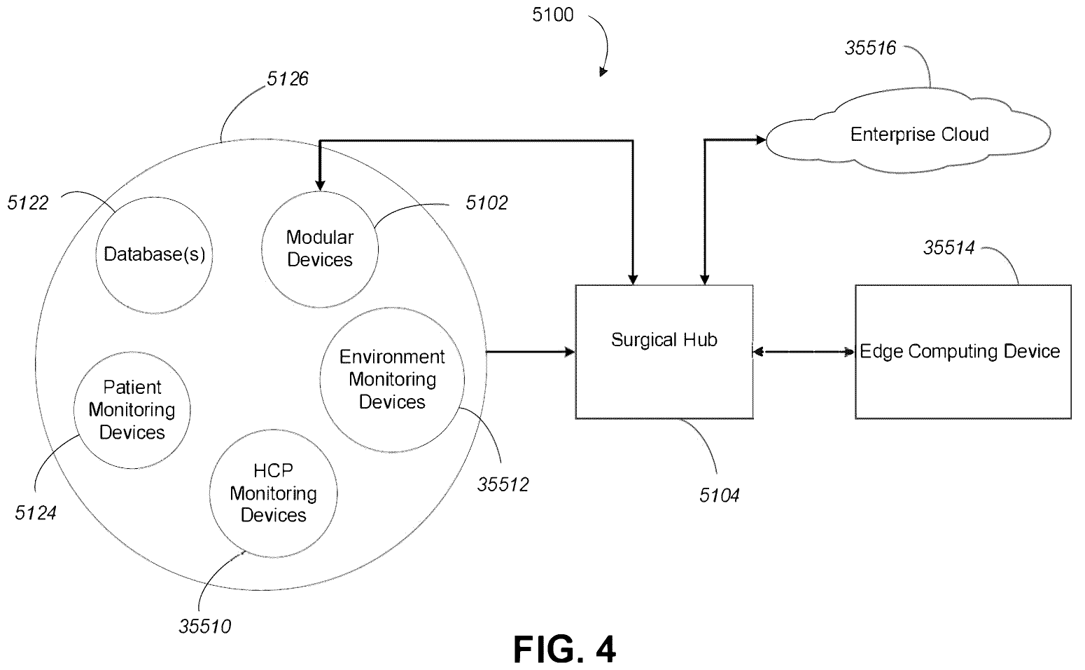

FIG. 4 illustrates a diagram of a situationally aware surgical system 5100. The data sources 5126 may include, for example, the modular devices 5102, databases 5122 (e.g., an EMR database containing patient records), patient monitoring devices 5124 (e.g., a blood pressure (BP) monitor and an electrocardiography (EKG) monitor), HCP monitoring devices 35510, and/or environment monitoring devices 35512. The modular devices 5102 may include sensors configured to detect parameters associated with the patient, HCPs and environment and/or the modular device itself. The modular devices 5102 may include one or more intelligent instrument(s) 20014. The surgical hub 5104 may derive the contextual information pertaining to the surgical procedure from the data based upon, for example, the particular combination(s) of received data or the particular order in which the data is received from the data sources 5126. The contextual information inferred from the received data can include, for example, the type of surgical procedure being performed, the particular step of the surgical procedure that the surgeon is performing, the type of tissue being operated on, or the body cavity that is the subject of the procedure. This ability by some aspects of the surgical hub 5104 to derive or infer information related to the surgical procedure from received data can be referred to as “situational awareness.” For example, the surgical hub 5104 can incorporate a situational awareness system, which may be the hardware and/or programming associated with the surgical hub 5104 that derives contextual information pertaining to the surgical procedure from the received data and/or a surgical plan information received from the edge computing system 35514 or an enterprise cloud server 35516. The contextual information derived from the data sources 5126 may include, for example, what step of the surgical procedure is being performed, whether and how a particular modular device 5102 is being used, and the patient's condition.

The surgical hub 5104 may be connected to various databases 5122 to retrieve therefrom data regarding the surgical procedure that is being performed or is to be performed. In one exemplification of the surgical system 5100, the databases 5122 may include an EMR database of a hospital. The data that may be received by the situational awareness system of the surgical hub 5104 from the databases 5122 may include, for example, start (or setup) time or operational information regarding the procedure (e.g., a segmentectomy in the upper right portion of the thoracic cavity). The surgical hub 5104 may derive contextual information regarding the surgical procedure from this data alone or from the combination of this data and data from other data sources 5126.

The surgical hub 5104 may be connected to (e.g., paired with) a variety of patient monitoring devices 5124. In an example of the surgical system 5100, the patient monitoring devices 5124 that can be paired with the surgical hub 5104 may include a pulse oximeter (SpO2 monitor) 5114, a BP monitor 5116, and an EKG monitor 5120. The perioperative data that is received by the situational awareness system of the surgical hub 5104 from the patient monitoring devices 5124 may include, for example, the patient's oxygen saturation, blood pressure, heart rate, and other physiological parameters. The contextual information that may be derived by the surgical hub 5104 from the perioperative data transmitted by the patient moni-toring devices 5124 may include, for example, whether the patient is located in the operating theater or under anesthesia. The surgical hub 5104 may derive these inferences from data from the patient monitoring devices 5124 alone or in combination with data from other data sources 5126 (e.g., the ventilator 5118).

The surgical hub 5104 may be connected to (e.g., paired with) a variety of modular devices 5102. In one exemplification of the surgical system 5100, the modular devices 5102 that are paired with the surgical hub 5104 may include a smoke evacuator, a medical imaging device such as the imaging device 20030 shown in FIG. 2, an insufflator, a combined energy generator (for powering an ultrasonic surgical instrument and/or an RF electrosurgical instrument), and a ventilator.

The perioperative data received by the surgical hub 5104 from the medical imaging device may include, for example, whether the medical imaging device is activated and a video or image feed. The contextual information that is derived by the surgical hub 5104 from the perioperative data sent by the medical imaging device may include, for example, whether the procedure is a VATS procedure (based on whether the medical imaging device is activated or paired to the surgical hub 5104 at the beginning or during the course of the procedure). The image or video data from the medical imaging device (or the data stream representing the video for a digital medical imaging device) may be processed by a pattern recognition system or a machine learning system to recognize features (e.g., organs or tissue types) in the field of view (FOY) of the medical imaging device, for example. The contextual information that is derived by the surgical hub 5104 from the recognized features may include, for example, what type of surgical procedure (or step thereof) is being performed, what organ is being operated on, or what body cavity is being operated in.

The situational awareness system of the surgical hub 5104 may derive the contextual information from the data received from the data sources 5126 in a variety of different ways. For example, the situational awareness system can include a pattern recognition system, or machine learning system (e.g., an artificial neural network), that has been trained on training data to correlate various inputs (e.g., data from database(s) 5122, patient monitoring devices 5124, modular devices 5102, HCP monitoring devices 35510, and/or environment monitoring devices 35512) to corresponding contextual information regarding a surgical procedure. For example, a machine learning system may accurately derive contextual information regarding a surgical procedure from the provided inputs. In examples, the situational awareness system can include a lookup table storing pre-characterized contextual information regarding a surgical procedure in association with one or more inputs (or ranges of inputs) corresponding to the contextual information. In response to a query with one or more inputs, the lookup table can return the corresponding contextual information for the situational awareness system for controlling the modular devices 5102. In examples, the contextual information received by the situational awareness system of the surgical hub 5104 can be associated with a particular control adjustment or set of control adjustments for one or more modular devices 5102. In examples, the situational awareness system can include a machine learning system, lookup table, or other such system, which may generate or retrieve one or more control adjustments for one or more modular devices 5102 when provided the contextual information as input.

For example, based on the data sources 5126, the situationally aware surgical hub 5104 may determine what type of tissue was being operated on. The situationally aware surgical hub 5104 can infer whether a surgical procedure being performed is a thoracic or an abdominal procedure, allowing the surgical hub 5104 to determine whether the tissue clamped by an end effector of the surgical stapling and cutting instrument is lung (for a thoracic procedure) or stomach (for an abdominal procedure) tissue. The situationally aware surgical hub 5104 may determine whether the surgical site is under pressure (by determining that the surgical procedure is utilizing insufflation) and determine the procedure type, for a consistent amount of smoke evacuation for both thoracic and abdominal procedures. Based on the data sources 5126, the situationally aware surgical hub 5104 could determine what step of the surgical procedure is being performed or will subsequently be performed.

The situationally aware surgical hub 5104 could determine what type of surgical procedure is being performed and customize the energy level according to the expected tissue profile for the surgical procedure. The situationally aware surgical hub 5104 may adjust the energy level for the ultrasonic surgical instrument or RF electrosurgical instrument throughout the course of a surgical procedure, rather than just on a procedure-by-procedure basis.

In examples, data can be drawn from additional data sources 5126 to improve the conclusions that the surgical hub 5104 draws from one data source 5126. The situationally aware surgical hub 5104 could augment data that it receives from the modular devices 5102 with contextual information that it has built up regarding the surgical procedure from other data sources 5126.

The situational awareness system of the surgical hub 5104 can consider the physiological measurement data to provide additional context in analyzing the visualization data. The additional context can be useful when the visualization data may be inconclusive or incomplete on its own.

The situationally aware surgical hub 5104 could determine whether the surgeon (or other HCP(s)) was making an error or otherwise deviating from the expected course of action during the course of a surgical procedure. For example, the surgical hub 5104 may determine the type of surgical procedure being performed, retrieve the corresponding list of steps or order of equipment usage (e.g., from a memory), and compare the steps being performed or the equipment being used during the course of the surgical procedure to the expected steps or equipment for the type of surgical procedure that the surgical hub 5104 determined is being performed. The surgical hub 5104 can provide an alert indicating that an unexpected action is being performed or an unexpected device is being utilized at the particular step in the surgical procedure.

The surgical instruments (and other modular devices 5102) may be adjusted for the particular context of each surgical procedure (such as adjusting to different tissue types) and validating actions during a surgical procedure. Next steps, data, and display adjustments may be provided to surgical instruments (and other modular devices 5102) in the surgical theater according to the specific context of the procedure.

Systems, methods, and instrumentalities associated with inter-connectivity of data flows between various surgical devices and/or systems are disclosed. The surgical devices and/or surgical systems may be interrelated or independent smart surgical devices and/or surgical systems. Data sourced by a first surgical device/system may be communicated to and/or accessible by a second surgical device/system for interactive use and storage. The data exchange may be bi-directional or unidirectional, which, for example, may enable the surgical devices/systems to interface and use each other's data. The data exchange may affect one or multiple surgical device/system's operation.

For example, a first surgical system may operate using a first operation configuration (e.g., first operation configuration parameters). The first surgical system may determine capability information associated with a surgical environment (e.g., operating room (OR)). The surgical environment may include surgical systems (e.g., surgical hub, surgical devices, etc.). The capability information may include information associated with what a surgical system may be capable of generating and/or providing. The first surgical system may receive first data and associated metadata (e.g., first metadata) from a second surgical system. The first metadata may indicate whether the first data is control data or response data (e.g., which portion of the first data is control data or response data). The first surgical system may select an operation configuration (e.g., operation configuration parameter) based on the first data and/or first metadata. For example, the first surgical system may determine the first operation configuration if the first data is response data. The first surgical system may determine the second operation configuration if the first data is control data. The first surgical system may generate second data based on the determined operation configuration. The first surgical system may determine data packages (e.g., including at least a portion of the second data) to send to target systems. The data packages may indicate whether the data in the data package is control data or response data.

For example, the first surgical system may determine capability information associated with the surgical environment based on a discovery procedure. The discovery procedure may include determining the surgical systems present or used in a surgical environment. The discovery procedure may include determining capabilities associated with each surgical system present or used in the surgical environment. The discovery procedure may be performed before determining an operating configuration to use. The first surgical system may determine capability information based on a pre-configuration (e.g., checklist, boot-up sequence). The pre-configuration may include information indicating surgical systems and their respective capabilities associated with the surgical environment.

The first surgical system may determine that received data is inaccurate and/or incomplete based on the received first data and the determined capability information. For example, the first surgical system may determine that a data or data type is missing. The first surgical system may send an indication indicating that the data is missing or the surgical system that generated and sent the data (e.g., second surgical system) is not operating properly.

Interrelated surgical systems may communicate and send data between each other. Systems that communicate with each other to share data may use the data to impact a receiving system's operation (e.g., function, processing, and/or the like). For example, the data may impact the operating configuration of a system. Data generated at a first surgical system may be sent to a second surgical system. The data received at the second surgical system may be used to modify the second surgical system's operating configuration (e.g., from a first operating configuration to a second operating configuration). This may lead to a detrimental cascade of feedback affecting functionality. For example, the second surgical system may generate data based on the updated operating configuration and send the data back to the first surgical system, which may then operate its operating configuration based on the return data. The detrimental feedback loop would continue to have the surgical systems altering their operating configuration based on continually updated parameters for generating data.

Interrelated systems that communicate and share data that may impact an operating configuration may use a data exchange framework to address the cascading feedback. Avoiding the cascading feedback may enable the systems to operate accurately.

A data exchange framework to address the cascading feedback may include data and/or metadata indicating whether the data is control data or response data (e.g., control indication or response indication). Metadata may be generated for data produced by a surgical system. The data and/or metadata may indicate whether the associated data is response or control data. Response data may indicate that a receiving surgical system should not modify its operating configuration based on the data Control data may indicate that the receiving surgical system may adapt its operating configuration (e.g., operating parameters) based on the received data.

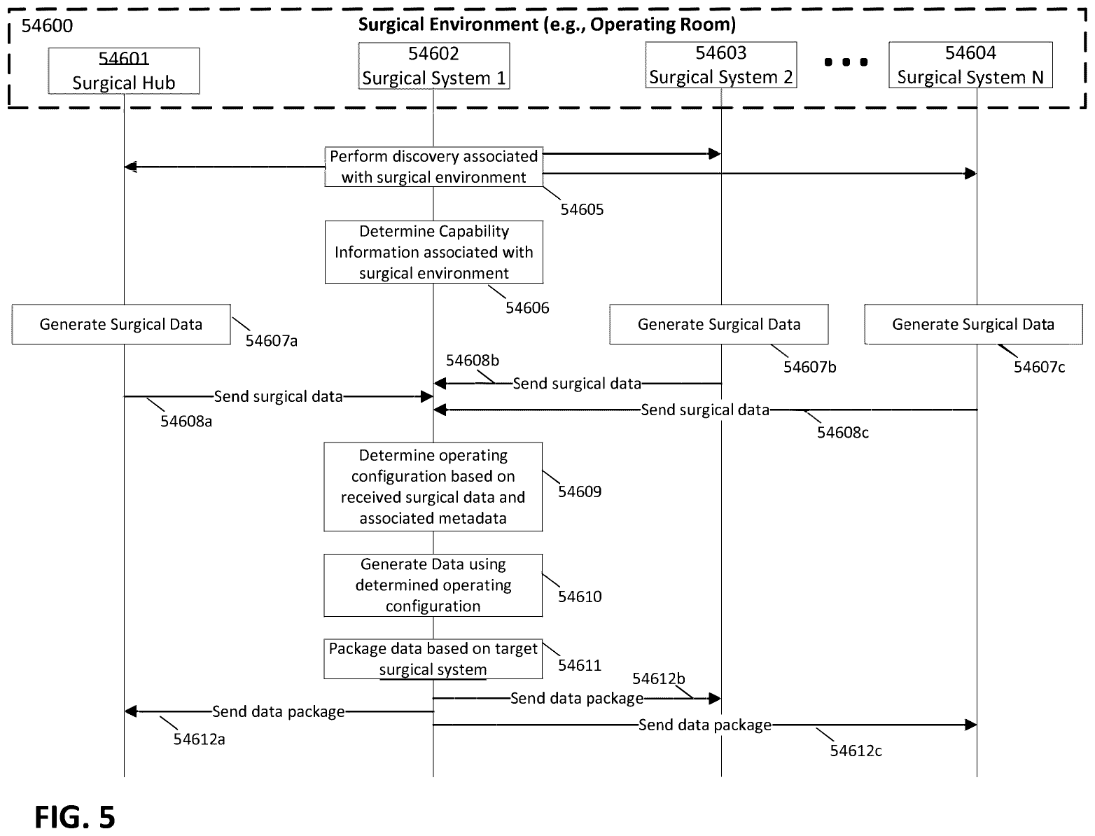

FIG. 5 illustrates an example flow of interrelated surgical systems exchanging data within a surgical environment. The surgical environment 54600 (e.g., operating room or theater, or medical facility) may include a surgical hub 54601 and a plurality of surgical systems (e.g., surgical system 1 54602, surgical system 2 54603, surgical system N 54604, etc.) The surgical system(s) may be interrelated and may generate data and/or communicate with each other (e.g., send data, such as, for example, control data or response data). The surgical system(s) may determine capability information associated with other systems in the surgical environment. The surgical system(s) may operate using configured operating configurations. The operating configurations may be changed and/or modified. The operating configurations may be changed based on data (e.g., generated data and/or data received from other systems). Surgical systems may generate data and/or perform surgical operations and/or actions according to an updated operating configuration.

As shown in FIG. 5, at 54605, discovery associated with the surgical environment may be performed. Discovery may be performed by surgical systems (e.g., surgical hub) in the surgical environment (e.g., before determining an operating configuration to use). The discovery may include determining the surgical systems present and/or being used in the surgical environment. The discovery may include determining the number of surgical devices and/or surgical systems in the surgical environment. The discovery may be performed by communicating with the surgical systems in the surgical environment. The discovery may be performed based on data received by the surgical hub in the surgical environment.

For example, discovery may be performed in association with a surgical environment start up (e.g., boot up process). A surgical environment may be set up to perform a surgical procedure. A surgical environment may be set up based on the surgical procedure. For example, a colorectal surgical procedure may be associated with using a first surgical system (e.g., a laparoscopic surgical system) and a second surgical system (e.g., an endoscopy surgical system), and a cardiovascular surgical procedure may be associated with the second surgical system and a third surgical system (e.g., a surgical system with energy device). The surgical systems used in the surgical environment may depend on the surgical procedure, patient, and/or healthcare professionals performing the surgical procedure (e.g., surgeon may have preferences for using specific surgical systems and/or tools). In examples, a checklist for surgical systems used in a surgical procedure may be used. The checklist may be used to perform discovery of the surgical systems in the surgical environment.

Discovery may include scanning a surgical environment based on a surgical procedure checklist and/or performing a dynamic discovery process associated with determining which surgical systems and/or tools are present in the surgical environment. The dynamic discovery process may validate the surgical procedure checklist, for example, to confirm whether a specific surgical system is present. The dynamic discovery process may validate whether a surgical system is properly configured (e.g., generating the proper data and/or able to communicate the data). The discovery process may validate that surgical systems within the surgical environment are operating correctly.

As shown in FIG. 5 at 54606, capability information associated with the surgical environment may be determined. The capability information may be determined for each surgical system and/or surgical hub in the surgical environment. Each surgical system and/or surgical hub may be associated with different capabilities (e.g., types of data produced, types of processing performed, and/or the like). For example, a first surgical system may be associated with a first capability and a second surgical system may be associated with a second capability. The first capability of the first surgical system may be associated with generating a first type of data, a second type of data, and/or a third type of data. The second capability of the second surgical system may be associated with generating the third type of data and/or a fourth type of data. The capabilities of the surgical systems may be used to determine whether or not appropriate data is being generated and/or communicated between systems.

For example, a first surgical system may be determined to be capable of generating and communicating a first, second, and third type of data (e.g., indicated in capability information). A surgical hub or second surgical system may receive the first and second type of data from the surgical system. The surgical hub or second surgical system may determine that the third type of data is missing from the first surgical system, for example, based on the determined capability information.

As shown in FIG. 5 at 54607a, 54607b, and 54607c, the surgical systems and/or surgical hub may generate data (e.g., according to their respective operating configurations). The generated data may be sent between the interrelated systems and/or the hub. For example, as shown at 54608a, 54608b, and 54608c, the first surgical system may receive data and/or respective metadata sent from the other surgical systems and/or surgical hub. The first surgical system may use the received data and metadata to perform according to its current and/or default operating configuration. The first surgical system may use the received data and metadata in determining which operating configuration to apply (e.g., whether to use the currently used operating configuration or switch operating configurations).

Metadata may provide context to bi-directional exchanged data. The metadata may provide context, for example, to enable (e.g., allow) bracketing usage implications for one or multiple systems. A bi-directional data feed may include an originating system utilization of the feed and a priority of the feed to a system's primary function.

Determination and/or communication of which data streams are being used as control streams and which ones are response streams may be performed. Systems may share data streams, where some of the data streams may be interrelated originating from different systems. Each system may use the data it is receiving to either control an aspect of its own function (e.g., operation) monitor for leading indicators of upcoming changes, or verify changes by monitoring the responses of the related systems. For example, if two systems both used feeds from each other that are interrelated, both as control feeds, there may be a detrimental cascade where each device may keep changing based on the received data and the change is monitored by the other system to in turn make another change that then may initiate a third change back at the first system. The detrimental cascade of feedback may be overcome if the bi-directional data exchanged indicated in metadata whether the feed is a driving control data feed or if it is a monitored response feed.

Systems may indicate which data streams are needed and the intended use of the data streams. The intended use may be included in metadata. For example, intended use may be indicated (e.g., via a notification of intended use). Systems may determine what data types are used by a different system to control an active element of operation. The notification of intended use may prevent both systems treating interrelated data-feeds as controlling. Relational determination of open and close loop control may be performed between two systems that are interrelated or interdependent on convoluted data streams. For example, one system of two systems may deactivate or operate in an open loop operation. The other system may be a controlling system.

Smart surgical systems may exchange information bi-directionally that each system may use or react to. Datasets may be non-interactive, interactive, or convoluted.

Non-interactive data sets may include a hub-to-smoke evacuator module bi-directional data flow, which may be independent uni-directional from each system to the other. A smoke evacuator may indicate a power consumption rate to another system for power management of the interrelated systems sharing the same power source. Smart systems (e.g., a hub or mantle backplane) may communicate power levels, hand piece energy density/duration (e.g., likelihood of causing smoke), energized status of generator, etc.

A convoluted data set may include data associated with smart controlled irrigation for advanced monopolar and/or bipolar cooling. The convoluted data may be exchanged between smart controlled irrigation devices and a generator. A smart irrigation device may receive power level or electrode temperature and activation status from a generator to control saline flow-rate, saline pressure, and/or saline temperature. The controlled rates may be communicated to the generator for adaptation of the control operations (e.g., algorithms). For example, the generator may receive a saline flow-rate, pressure, fluid temperature, and/or the like, to control power level, constant current versus power, energy modality, etc. The generator may control the system under normal parameters and allow the smart irrigation system to adjust, or the smart irrigation system may be predefined and the generator may adjust to minimize charring and/or tissue sticking. Both systems may be enabled to adjust some (e.g., not all) of the control parameters each using (e.g., relying) on the other system to adjust the other impacting parameters.

In examples a generator (e.g., a first system) and an energy device (e.g., a second system) may interact. The energy device may be determined to have a resistance over time below a nominal threshold (e.g., cutting or sealing too fast). The generator may react by changing the power level for the next activation. A third system (e.g., graspers/robotic arm), which covertly reviewed the data and sees the data transfer and changes applied between the two systems, may determine that the tension and/or load that is on the tissue when fired is too much based on the data transferred. The third system may alter the position at which it is holding the tissue, for example, to minimize the tension when cutting and/or sealing.

Translation ability and/or the ability to understand data in output form (e.g., raw form) may be enabled. Metadata head or cipher may be included in data, for example, to define format, language, relational organization of the data, and/or the like (e.g., to enable interpretation of the data). For example, a patient monitor device may collect biomeasures associated with a patient (e.g., PO2, CO2, EKG, blood pressure, core temperature, etc.) but may not have dedicated real-time output ports for the measures. The patient monitor device may use a single output port with a combined data stream. The monitors may not be collected using a sampling rate. The data stream may vary point by point of what data is being exported. Metadata cipher may be used to determine what measures are within a data point or may provide a format or identifier as to which data points allow an external system to decompose one stream into independent steams.

As shown in FIG. 5 at 54609, the first surgical system may determine an operating configuration based on the received surgical data and/or associated metadata. The first surgical system may be operating using a first operating configuration (e.g., first operating configuration parameter). The first surgical system may change the applied operating configuration (e.g., from the first operating configuration to a second operating configuration). The change in applied operating configuration may be determined based on data, for example generated by the first surgical system and/or received data associated with other surgical systems and/or hubs.

The first surgical system may determine which operating configuration to apply based on a classification associated with received data (e.g., data generated by another system or hub). For example, received data may be control data (e.g., data indicating to control the system receiving the data) or response data (e.g., data indicating that the data is generated in response to an aspect associated with the first surgical system).

For example, data may be control data and sent to the first surgical system to affect the operating configuration. For example, first data received by the first surgical system may be control data and the first surgical system may determine operating parameters and/or a suitable operating configuration based on the received control data. The control data indicates that the first surgical system may modify its operating configuration as needed.

For example, data may be response data and sent to the first surgical system. The response data may be generated by a second surgical system. The second surgical system may have been operating using a third operating configuration and may have switched to a fourth operating configuration based on data the second surgical system received (e.g., control data sent by the first surgical system to the second surgical system). The second surgical system may generate data based on the fourth operating configuration and send the data as response data to the first surgical system. The first surgical system may determine that the data received is response data and may refrain from modifying its operating configuration based on the data being response data.

The classification of control data and response data may prevent a cascading feedback problem between communicating surgical systems. For example, if there is no indication of control or response data, systems may continually change their operating configurations and generate data accordingly based on incoming received data. For example, a first surgical system may send data to a second surgical system. The second surgical system may update its operating configuration from a first operating configuration to a second operating configuration based on the data received from the first surgical system. Then the second surgical system may generate data based on the second operating configuration and send the data to the first surgical system. The data received that was generated by the second surgical system using the second operating configuration may impact the operating configuration of the first surgical system. For example, the first surgical system may then alter its operating configuration from a third operating configuration to a fourth operating configuration and generate data using the fourth operating configuration. The data generated by the first surgical system using the fourth operating configuration may be sent to the second surgical system. The second surgical system may continue the cascading feedback loop and continually update the operating configuration. The cascading feedback loop would negatively impact the operation of the surgical systems.

The control data and response data classification may prevent the cascading feedback loop. For example, a surgical system receiving control data may be indicated to update its configuration based on the control data. A surgical system receiving response data may be indicated to refrain from updating its configuration based on the response data. The response data may act as a termination of the cascading feedback loop.

As shown in FIG. 5 at 54610, the first surgical system may generate surgical data (e.g., surgical procedure data, surgical equipment data, patient information data, OR information, facility information, surgical system data, surgical event data, and/or the like) using the determined operating configuration. The generated data may be in response to control data received by the first surgical system. The first surgical system may send its generated data, for example, to the interrelated systems in the surgical environment. As shown at 54611, the first surgical system may determine data packages to send to respective interrelated systems. As shown at 54612a, 54612b, and 54612c, the data packages may be sent to the respective surgical systems. The data packages may indicate whether the data in the respective data packages are control data or response data.

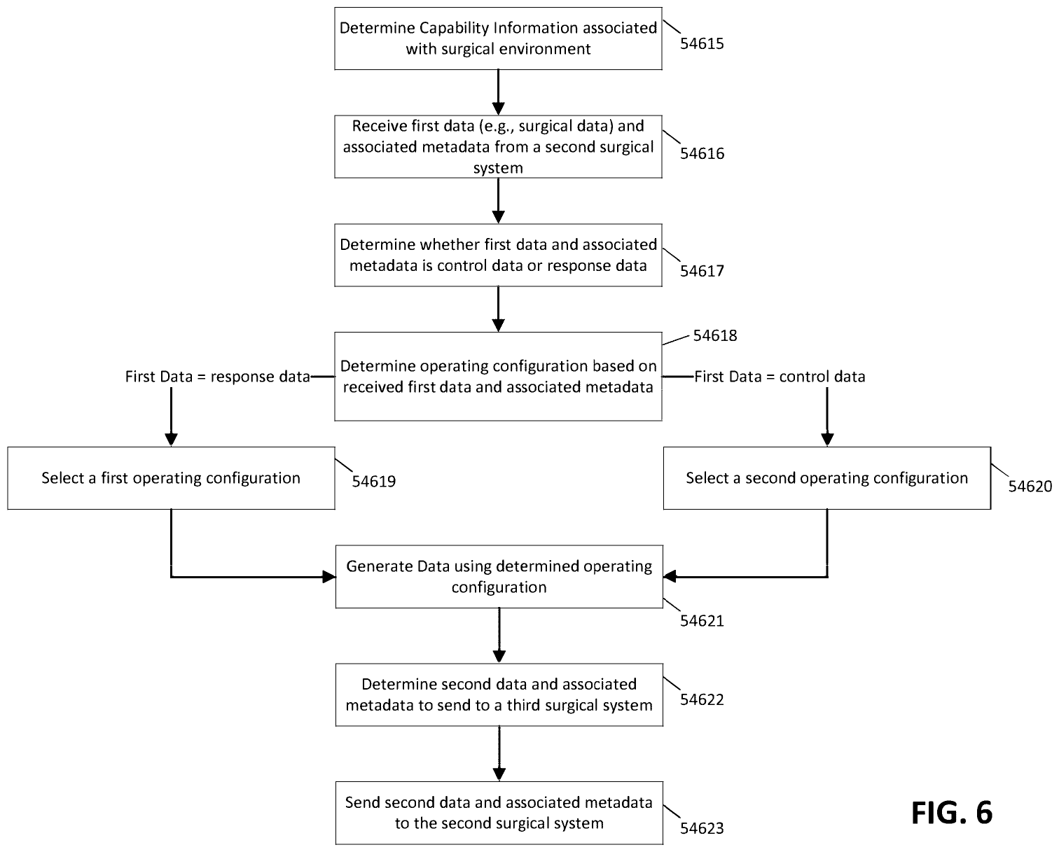

FIG. 6 illustrates an example flow of determining an operating configuration based on received data and/or associated metadata. A first surgical system may determine an operating configuration (e.g., determine whether to maintain a current operating configuration or apply a different operating configuration) based on received data (e.g., based on whether received data is control data or response data). The first surgical system may determine to modify its operating configuration if the received data is control data. The first surgical system may determine to maintain its current operating configuration if the received data is response data. The first surgical system may generate data based on the applied operating configuration after receiving the initial data.

As shown at 54615, the first surgical system may determine capability information associated with a surgical environment. The capability information may indicate the surgical systems and/or surgical devices that may be communicated with and/or interrelated in the surgical environment. The capability information may indicate the types of data the surgical systems in the surgical environment are capable of generating and communicating. Surgical systems may use the capability information to determine whether another surgical system sending data is a closed loop or open loop system. The capability information may indicate whether another surgical system sends control data or response data.

As shown at 54616, the first surgical system may receive first data and associated metadata from a second surgical system. The first data and associated metadata may indicate whether the first data is control data or response data. As shown at 54617, the first surgical system may determine whether the first data and associated metadata is control data or response data.

As shown at 54617, the first surgical system may determine an operating configuration based on the received first data. For example, if the first data is control data, the first surgical system may determine whether to modify its operating configuration based on the first data. If the first data is response data, the first surgical system may refrain from considering the data to determine its operating configuration.

As shown at 54619, the first data may be response data. The first surgical system may be operating using a first operating configuration (e.g., when it received the first data). Because the first data is determined to be response data, the first surgical system may determine to continue using the first operating configuration (e.g., refrain from switching operating configurations and/or parameters).

As shown at 54620, the first data may be control data. The first surgical system may be operating using the first operating configuration (e.g., when it received the first data). Because the first data is determined to be control data, the first surgical system may determine to consider the first data in determining its operating configuration (e.g., whether to maintain the first operating configuration or switch to a second operating configuration). For example, the first surgical system may determine to apply a second operating configuration based on the first data.

As shown at 54621, the first surgical system may generate data based on the applied operating configuration. The generated data may be sent to surgical systems (e.g., as described herein with respect to FIG. 5). For example, a third surgical system may be determined. The first surgical system may determine (e.g., generate) second data and associated metadata to send the second surgical system (e.g., as shown at 54622). The second data may be generated based on the applied operating configuration. Metadata may be generated and/or determined that is associated with the second data. For example, the metadata associated with the second data may indicate whether the second data is control data or response data. Accordingly, the first surgical system may send the determined second data and associated metadata to the second surgical system (e.g., as shown at 54623).

In examples, a first surgical system may operate using a first operating configuration. The first surgical system may determine capability information associated with a surgical environment. The first surgical system may receive surgical environment information that indicates surgical system information associated with a default surgical system environment (e.g., for a specified surgical procedure). The first surgical system may perform a discovery operation associated with the surgical environment. The first surgical system may determine surgical environment information based on the performed discovery. The capability information may include the default surgical system environment and the discovered surgical environment information. The capability information may indicate surgical system information (e.g., including a list of surgical systems associated with a surgical environment). The capability information may indicate the data (e.g., type of data) that a surgical system is capable of generating, measuring, communicating, and/or providing. The first surgical system may receive first data from a second surgical system (e.g., indicated in the surgical system information. The first data may include first metadata that may indicate that the first data is control data or response data. The first surgical system may select an operating configuration based on the first metadata (e.g., a first operating configuration or a second operating configuration). The first surgical system may select the first operating configuration (e.g., maintain the first operating configuration) if the first metadata indicates that the first data is response data. The first surgical system may select the second operating configuration if the first metadata indicates that the first data is control data. The first operating configuration may be associated with the first surgical system operating using a first operating parameter and the second operating configuration may be associated with the first surgical system operating using a second operating parameter. The second operating parameter and/or second operating configuration may be determined based on the first data. The first surgical system may generate second data based on the selected operation configuration. The first surgical system may determine a third surgical system in the list of surgical systems associated with the surgical environment to send at least a portion of the second data (e.g., where the third surgical system is the same system as the second surgical system). For example, the first surgical system may determine that the third surgical system is to receive a portion of the second data. The first surgical system may send (e.g., via a data stream) the portion of the second data, for example, to the third surgical system. The data stream may include second metadata that may indicate that at least a portion of the second data is control data or response data.

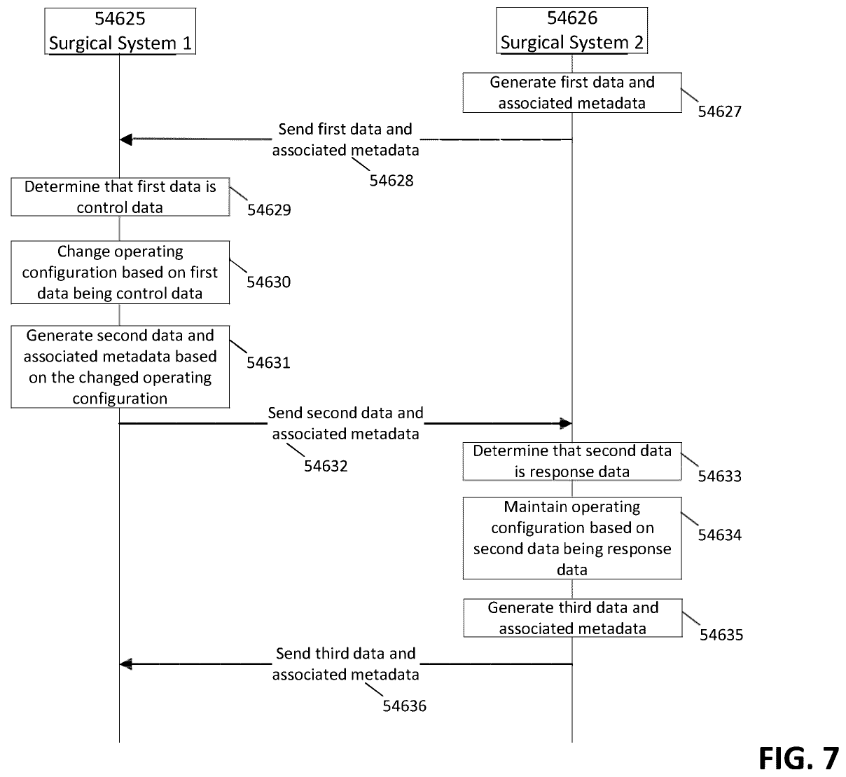

FIG. 7 illustrates an example flow of determining an operating configuration based on received data. As illustrated in FIG. 7, a first surgical system (Surgical System 1, 54625) may be interrelated with a second surgical system (Surgical System 2, 54626). The interrelated surgical systems may share data between each other. The interrelated surgical systems may modify their operating configuration based on received data from each other.

As shown at 54627, the second surgical system may generate first data and associated metadata (e.g., as described herein). The first data may be surgical data. That first data may be determined to be control data or response data. For example, surgical control data may be used for controlling a surgical system. The first data may be control data if first data is intended to be considered by the receiving surgical system (e.g., first surgical system) in determining an operating configuration that may be used by the receiving surgical system. The first data may be response data if the first data is intended to be refrained from being considered (e.g., not be considered) by the first surgical system in determining an operating configuration. As shown at 54628, the second surgical system may send the first data and associated metadata to the first surgical system.

As shown at 54629, the first surgical system may determine that the first data is control data. Based on the first data being control data, the first surgical system may consider the control data in determining an operating configuration to use. For example, the first surgical system may be using a first operating configuration before receiving the first data and associated metadata. The first surgical system may determine whether to change the operating configuration based on the first data. For example, the control data may not warrant changing the first surgical system's operating configuration. In this case, the first surgical system may determine to refrain from changing the operating configuration.

In examples, the first surgical system may determine to change the operating configuration to a second operating configuration (e.g., as shown at 54630). The determination to change the operating configuration may be based on the first data being control data and/or the contents of the first data.

As shown at 54631, the first surgical system may generate second data and associated metadata based on the changed operating configuration. The first surgical system may send the second data and associated metadata, for example, to the second surgical system (e.g., as shown at 54632). The second data and associated metadata may indicate that it is response data (e.g., in response to the control data received from the second surgical system), for example, to prevent a cascading feedback loop.

The second surgical system may determine that the second data is response data (e.g., as shown at 54633), for example, based on the second data and/or associated metadata. The second surgical system may determine to refrain from considering the second data in determining an operating configuration. For example, the second surgical system may determine to maintain its currently applied operating configuration (e.g., based on the second data being response data), as shown at 54634. Accordingly, the second surgical system may generate third data and associated metadata based on the maintained operating configuration (e.g., as shown at 54635). The second surgical system may send the third data and associated metadata, for example, to the first surgical system (e.g., as shown at 54636). The third data and/or associated metadata may indicate whether the control data (e.g., if the second surgical system wants to update the operating configuration of the first surgical system) or response data (e.g., if the second surgical system wants the first surgical system to refrain from considering the third data in determining an operating configuration to use.

Interrelated systems may use and/or rely on other systems to provide complete data, for example, to properly function. For example, a first system that is capable of providing a first type of data and a second type of data may send the two types of data to a second system. The second system may use both types of data to operate correctly. If the first system only sends one of the two types of data (e.g., only the first type of data or only the second type of data), the second system may not be able to operate properly. In other examples, a system using incomplete or inaccurate data in its operation may operate incorrectly.

For example, a first system may expect first data, second data, and third data from a second system. The second system may have only sent the first data and the third data. Without the second data, the first system may not operate properly.

Capability information associated with the surgical environment may be leveraged to determine whether correct data is being communicated between interrelated systems. The capability information associated with the surgical environment may be determined (e.g., as described herein, specifically in reference to FIG. 5). The capability information of the surgical environment may be used (e.g., by a surgical system) to check that received data from a sending system is accurate and/or complete (e.g., includes all the data that the sending surgical system is capable of generating and/or sending). For example, the capability information may be used to determine that a surgical system is capable of providing first, second, and third data. A complete data set would include the first, second, and third data.

Based on the capability information and received data, whether received data is incorrect or incomplete may be determined. Based on a determination that the data is incorrect or incomplete, a receiving system may send an indication to the sending system (e.g., system that generated the incomplete and/or incorrect data). The indication may indicate that the data was incorrect and/or incomplete. The indication may indicate to correct the issue (e.g., send the missing data or correct the improper operation).

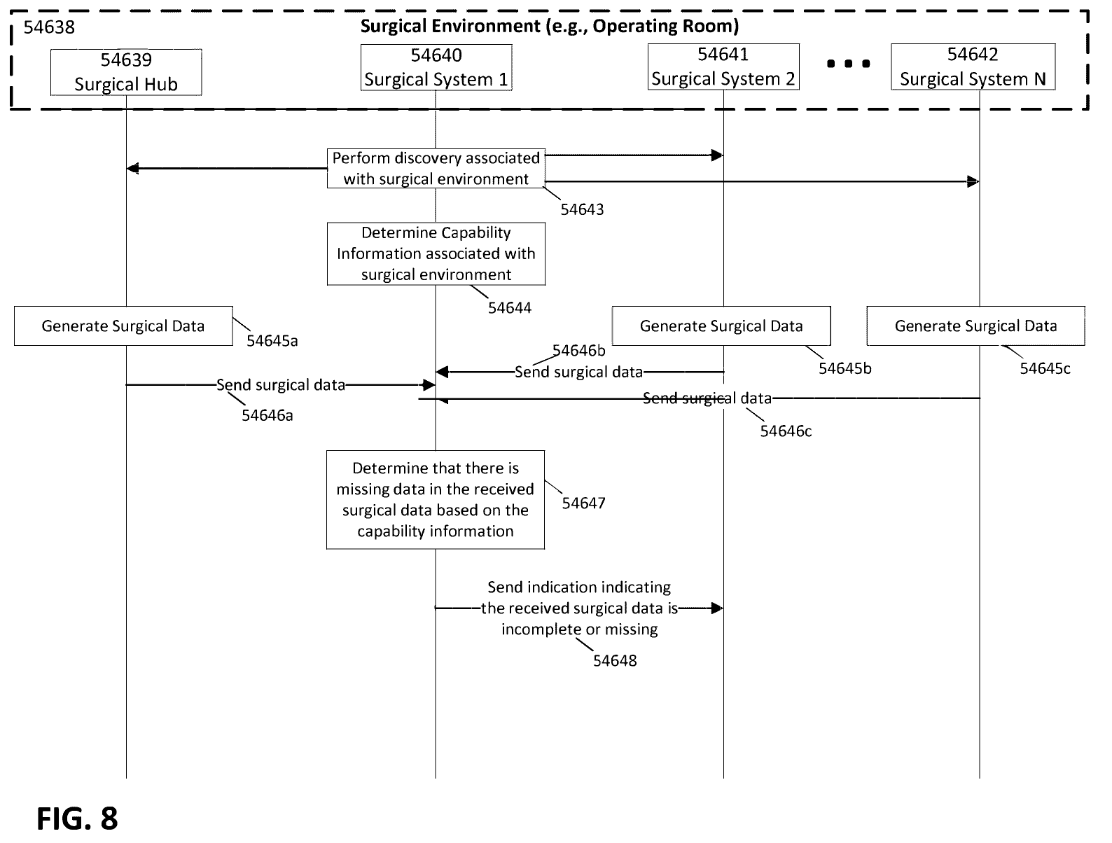

FIG. 8 illustrates an example flow of determining capability associated with a surgical environment and determining whether a dataset is complete and/or accurate. As shown in FIG. 8, a surgical environment 54638 may include surgical hub(s) (e.g., Surgical Hub 54639) and/or surgical system(s) (e.g., Surgical system 1 54640, surgical system 2 54641, and surgical system N 54642). The surgical systems and/or hubs may be interrelated (e.g., may send data between each other).

As shown at 54643, discovery associated with the surgical environment may be performed (e.g., by the first surgical system). The discovery may be performed to determine the surgical systems and/or hubs within the surgical environment, and/or determine the capability associated with the surgical systems and/or hubs in the surgical environment.

As shown at 54644, capability information associated with the surgical environment (e.g., capability information associated with respective surgical systems) may be determined (e.g., by the first surgical system). The capability information may indicate the capabilities associated with each surgical system within the surgical environment. For example, the capability information may indicate that a second surgical system is capable of providing first data (e.g., first type of data), second data (e.g., second type of data), and N data (e.g., Nth type of data). The capability information may indicate respective data and/or data types that surgical systems may be able to generate. For example, the surgical hub 54639 may generate data at 54645 according to the capability information associated with the surgical hub. Similarly, surgical system 2 54641 and surgical system N 54642 may generate data 54645b and 54645c respectively, for example, according to their respective capabilities indicated in the capability information. The generated surgical data may be sent, for example, as shown at 54646a, 54646b, and 54646c.

As shown at 54647, it may be determined that there is missing data in the received surgical data, for example, based on the capability information. In examples, the received data may not be complete or the data may be inaccurate. The completeness and/or accuracy of the data may be determined, for example, based on the capability information and received data. For example, the capability information may indicate that a first data type and a second data type may be received from a surgical system. It may be determined that received data is incomplete if it includes (e.g., only includes) the first type of data. The received data may be validated (e.g., cross-checked) with the capability information associated with a sending system.

A first surgical system may be configured to determine that first data received from a second surgical system is incomplete and/or inaccurate. The first surgical system may obtain capability information associated with the second surgical system (e.g., as described herein). The first surgical system may receive data from the second surgical system. The first surgical system may determine that the data received from the second surgical system is incomplete and/or inaccurate based on capability information associated with the second surgical system and the received data (e.g., contents of the received data). For example, the capability information may indicate that the second surgical system is capable of measuring and/or sending a first type of data and a second type of data, and the received data may include the first type of data (e.g., only the first type of data). The first surgical system may send an indication, for example, to the second surgical system. The indication may indicate to the second surgical system that the data is incomplete and/or inaccurate.

For example, the second surgical system may send processed data to the first surgical system. The first surgical system may desire to use raw, unprocessed data. Raw and/or unprocessed data may include surgical data that is sensed by a surgical system. The raw and/or unprocessed data may include surgical data that is not transformed. The first surgical system may indicate to the second surgical system to provide the unprocessed, raw data.

As shown at 54648, an indication may be sent. An indication may be sent to a surgical system that indicates that the previously sent data is incomplete and/or accurate. Based on a determination that received data is incomplete or inaccurate, the indication sent may indicate to the sending surgical system (e.g., an indication may be sent from surgical system 1 to surgical system 2 as shown in FIG. 8) that the data is incomplete or incorrect. The indication may further indicate to send the correct/appropriate data. The indication may further indicate to check the operating configuration to determine that the surgical system is operating as expected. The indication may further indicate to rectify issues associated with the sent data.

Real-time data exchange and processing may be performed. The real-time data exchange and processing may be associated with partitioned smart systems (e.g., surgical systems). Real-time exchange data may be partitioned. The real-time exchange data may be temporally partitioned. The temporally partitioned real-time exchange data may be used.

For example, smart systems out of sequence with a surgical procedure may determine that devices are capable of performing in the surgery operation. In examples, smart chargers for battery powered devices may be used. Smart chargers for battery powered staplers may communicate a status of the battery. The smart chargers may receive anticipated demand associated with the smart charger, a smart battery, the smart stapler, and a smart hub (e.g., surgical hub).

The smart charger may detect battery properties. For example, the smart charger may detect battery recharge capacity, status of current charge level, completion of charge timing, and/or the like. The battery properties may be communicated, for example, to other smart systems and/or the smart hub. The battery properties may be considered and/or used (e.g., by the hub) to define surgical procedure parameters (e.g., start/stop times, operating room throughput, etc.).

In examples, the data from the smart hub on utilization rate and/or timing may be used by the charger. For example, the smart charger may determine to switch between trickle charging (e.g., better for battery capacity). The smart charger may determine when to finish charging (e.g., maintaining the battery at a specified percentage (e.g., 80%) charge in maintenance mode. The smart charger may determine to complete the charging (e.g., just in time) for the procedure (e.g., which may be better for battery health longevity).

The smart charger may receive information associated with scheduling of surgical procedures and/or the next series of surgical procedures that may use a battery operated surgical device (e.g., power stapler and/or ultrasonic hand-held devices). The smart charger may determine charging parameters (e.g., charging speed, order of batteries being charged) based on the received information. The smart charger may determine charging parameters based on the usage information (e.g., usage for the surgical procedure). The smart charger may determine charging parameters (e.g., order of charging) to ensure that a fully charged battery is ready to be used at the time of each procedure. The smart charger may review the usage cycle of the batteries. The smart charger may charge a battery most appropriate for use based on balancing the usage between a set of batteries. The smart charger may determine to use all the charge of a first battery (e.g., intentionally), for example, to allow time for a replacement to be acquired without other batteries using a replacement simultaneously.

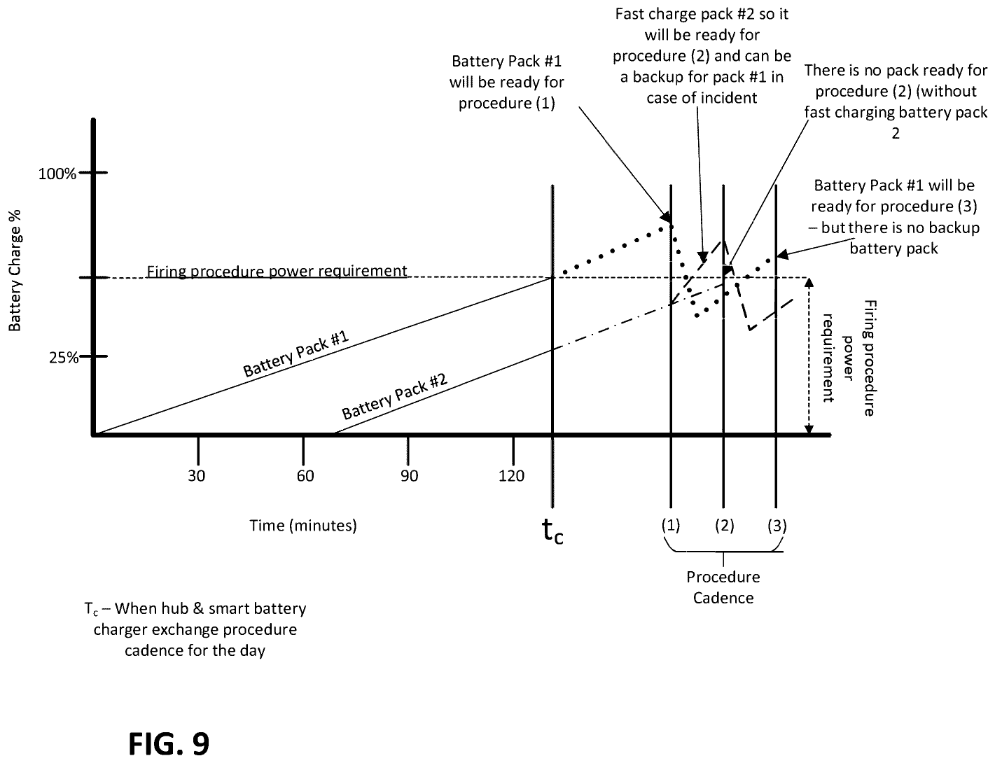

FIG. 9 illustrates an example of a smart battery charger sending usage information. As shown in FIG. 9, the smart battery charger for a powered stapler may have two batteries on the charge (e.g., at 25% charger and 55% charge respectively). The hospital surgical hub may inform the charger about the number of upcoming surgical procedures, the expected number of firings during each of those surgical procedures, and/or the timing of the firings, for example. The charger may inform the surgical hub when the first powered surgical stapler may be ready, and/or whether the charger will use fast charge mode for a second pack to be ready for a subsequent surgical procedure. The charger may send information indicating that the first battery pack will have sufficient charge for a first surgical procedure and a third surgical procedure (e.g., with a second procedure performed in between) that do not overlap in timing.

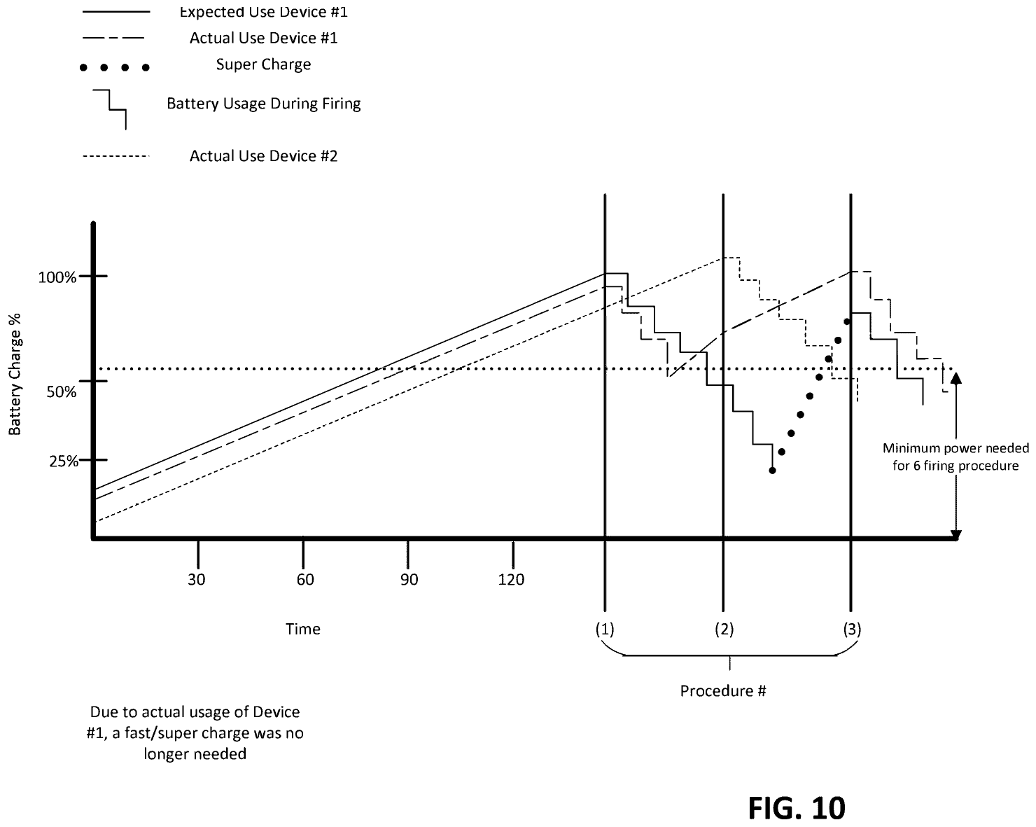

FIG. 10 illustrates an example of smart chargers for battery power stapling. As shown in FIG. 10, the smart charger may communicate with a smart hub and a surgical device (e.g., smart hand-held device). The smart charger may communicate with the smart hub regarding procedure plans, timing, cadence, and/or the like. The smart charger may communicate with the smart hand-held device regarding real-time updates on the battery levels and the consuming of the power. In the case of a procedure where there is less power used than expected (e.g., predicted), the smart charger may adjust charging rates and/or timing of the other batteries, for example, to balance the use of the sufficiently charged batteries with the desire to charge slowly (e.g., which may prolong the lifespan of the battery). As shown in FIG. 10, a power stapler may use (e.g., require) 50% of a charge for six 60 mm thick tissue firings in a gastric sleeve. A second device may be ready for a first procedure at 75% charge, and because three firings were used in the first surgical procedure, fast charging may be refrained from being performed (e.g., not be needed), and the second device may be ready for a second use for a third surgical procedure. A different battery may not use (e.g., require) a special charging for the three procedures to be handled by the two battery packs.

Real-time updates from the smart stapling device may be used by other surgical devices, for example, such as non-wired battery powered smart devices. The real-time updates may indicate a higher use of the battery or less use, which may impact the smart charger's charging parameters (e.g., cleaning and charging parameters for the data to support the usage needs). The real-time updates may enable a charger to monitor the rate at which the battery was discharged and/or the magnitude of the discharge, for example, to enable the smart charger to adjust its conditioning during recharging (e.g., to minimize effects on the battery chemistry for heavy use procedures). The conditioning may include a slow charge, a charge of a portion and holding for a period of time, a small incremental pining on/off charging (e.g., to induce the chemistry within the battery to re-balance after being heavily used or fully discharged), and/or the like. The conditioning may reduce the effects on the electrolyte mix (e.g., of the battery) and/or reduce event reverse corrosion or damage to the electrodes.

An event may be detected based on the communication between smart devices. For example, a dependent event may be detected and the detection may be confirmed. Confirmation of an action by a first system may be reflected in an output of an unconcerned system.

For example, biopsy needle penetration depth may be monitored (e.g., by ultrasound imaging of the tumor and needle). A robotic surgical system may change the depth of the needle and may use (e.g., require) confirmation that the current ultrasound image includes the updated motion before another motion is permitted. The robotic surgical system may monitor the ultrasound output to detect a related action to confirm the ultrasound stream is up to date before performing subsequent actions. The ultrasound may provide a metadata tag relating to time which the robot may use as a measure to ensure the action it took within a related timing is up to date.

Surgical procedure data (e.g., full surgical procedure data) from a smart system may be downloaded and/or processed. The download and/or processing of the full surgical procedure data may be accessible outside the surgical event (e.g., after the surgical procedure and/or event). The downloaded surgical data from a first surgical procedure and associated post completion and a second surgical procedure and associated post completion may be compiled and analyzed for updating control parameters (e.g., control program) of a smart device (e.g., before performing a third surgical procedure). For example, a hand-held ultrasonic device may not include a communication array for real-time transfer of data. The hand-held ultrasonic device may transfer surgical procedure information (e.g., usage information) after the surgical procedure or use. The device may transfer the information after a first surgical procedure, or after the first surgical procedure and a second surgical procedure. A hub or smart device may use the information (e.g., usage information) to determine an improvement for the hand-held ultrasonic device (e.g., improve the algorithm or operation). The hand-held device may receive feedback (e.g., during its charge cycle) for its operation in subsequent procedures.

Real-time data collection may enable real-time data usage. The real-time data may be collected. The real-time data may be transformed and/or used. The exchange of the real-time data (e.g., transformed and/or processed real-time data) may be exchanged between surgical systems (e.g., interrelated surgical systems).

The collected data may be transformed in real-time (e.g., a field programmable gate array (FPGA) may be used to collect the data). The transformed real-time data may be used (e.g., acted on) in a real-time flow form (e.g., during a surgical procedure. The organizational structure of the data may be used for a reaction to the data (e.g., using a predefined algorithm to use the data stream to control another portion of the system without understanding the meaning of the data or the trends).

Data may be exchanged and/or moved from electronic health records (HER) to a surgical system, for example, using a database field structure to determine what data streams belong where. Real-time data may be exchanged between control systems and smart systems (e.g., continuous data stream with no context). The real-time data may be used with predefined limits, rules, and/or based on specific conditions being satisfied (e.g., notifications and/or actions).

For example, a lack of signal may imply an issue with a sensor lead and/or a monitored parameter. Either issue may warrant a notification to the HCP. The system may determine that the measurement and/or data is outside specific bounds (e.g., thresholds). For example, an electrocardiogram (ECG) lead may produce data outside expected bounds, e.g., patient crashing or loss of adhesion of the ECG lead. Regardless of the issue, both issues may be brough to the HCP's attention to rectify. In examples of finger pulse oximetry data, data outside of expected bounds may not be urgent to rectify.

Raw data in real-time may be transformed, for example, to be used or acted on by other smart systems in real time. The meaning of the content of data may be understood, for example, to enable action and/or use of the data. For example, the information contained within transferred data may be represented using common language to understand the meaning of the content. Smart systems may be enabled to access and/or process data from multiple sources without losing meaning. The smart systems may integrate that data for mapping, visualization, and/or other forms of representation and analysis. Real-time data may be exchanged between advanced imaging or penetration imaging devices. For example, sharing the location of detected relevant items from a detecting system to a system that may be impacted by the location (e.g., because the detection of the metal may be transferred and highlighted) may imply that a system knows the meaning of the data and highlighting its implication to the system that may depend on the determined location.

For example, overlapping staples in a surgical procedure may be problematic. For an endocutter, staple locations may be problematic in one or more of the following: knife part through the metal; notch knife and reduce performance of knife, dragging staples along a length, grabbing buttress and dragging along. For a circular cutter or circular stapler, staple locations may be problematic in one or more of the following: multiple layers of tissue (e.g., compression selected based on 2 or 4 layers of tissue); circular firing through a linear line; blade damage via contact with metal.

Hidden metal via previous surgeries may be problematic, and may need to be determined for a surgical procedure. Adjacency of metal may be problematic for imaging and/or surgical devices (e.g., cutter, stapler, etc.). Instrument outcomes may be affected based on unknown metal being within an action portion of the jaws of a device. Still resistance during closing (e.g., of a stapler or cutter) hall effect sensor abnormalities, unusual rapid impedance drops, and/or the like may result from an unknown metal. Location of unknown metal may be used to prevent breaking a blade or a short out in the direct contact of an unanticipated piece of metal.

Unidirectional communication may be used to broadcast data known by one system to other interrelated systems. The data may be broadcasted to independent systems via encrypted unidirectional communication, for example.

For example, a surgical procedure step may be identified by a first smart system. The first smart system may broadcast the surgical procedure step to other smart systems. The other smart systems may use the data (e.g., if needed) or discard the data (e.g., if determined to not be useful). Multiple broadcast channels (e.g., with various levels of encryption) may be used and/or available to communicate with other smart systems. Unidirectional communication may be used to transfer data to smarter systems (e.g., from a system with limited capabilities to a system with comparably more capabilities). For example, a system may not be aware of what it is connected to based on a unidirectional nature (e.g., a first surgical system may not be aware that it is connected to a second surgical system). Broadcasting data may allow for a higher chance of sending data to a smart system that may use it (e.g., it may be useful for). A smart system may turn off (e.g., need to turn off) a system with unidirectional communication. Accordingly, the smart system may notify an HCP about a malfunctioning system. The smart system may remove power of to a system (e.g., malfunctioning system).

Systems, methods, and instrumentalities are disclosed associated with adaptation of operation associated with monitoring of surgical devices. A first surgical device may monitor operation of a second surgical device. The monitoring of the second surgical device may affect the operation of the first surgical device (e.g., impact data generation). The first surgical device or the second surgical device may change its operating configuration, for example, based on the monitoring.

For example, a first surgical system may operate using a first operating configuration (e.g., first parameter). The first surgical system may determine first data, for example, using the first operating configuration. The first surgical system may determine that it is being monitored (e.g., by a second surgical system), for example, based on the first data. The first data may be impacted based on the monitoring. The first surgical system may determine an impact associated with the surgical system being monitored (e.g., impact on the first data). The first surgical system may determine an operating configuration to use based on the impact. The first surgical system may compare the impact with a threshold. For example, the first surgical system may determine to use (e.g., maintain) the first operating configuration if the impact is less than the threshold. For example, the first surgical system may determine to use (e.g., change to) a second operating configuration (e.g., second parameter) if the impact is greater than or equal to the threshold. The first surgical system may operate (e.g., generate second data) using the determined operating configuration.

For example, a first surgical system may monitor a second surgical system. The first surgical system may try to remain undetected by the second surgical system. The first surgical system may use a first operating configuration, for example, to monitor the second surgical system. The first surgical system may obtain first data (e.g., via the monitoring) associated with the second surgical system. The first surgical system may determine an impact associated with the second surgical system being monitored. The impact may be determined based on the first data. The first surgical system may determine an operating configuration to use based on the impact associated with the second surgical system being monitored. For example, the first surgical system may compare the impact with a threshold. For example, the first surgical system may determine to use (e.g., maintain) the first operating configuration if the impact is less than the threshold. For example, the first surgical system may determine to use (e.g., change to) a second operating configuration if the impact is greater than or equal to the threshold. The first surgical system may operate (e.g., monitor the first surgical system and/or obtain second data associated with the second surgical system) using the determined operating configuration.

Interrelated systems may exchange data. The interrelated systems may be located within a surgical environment, surgical facility, hospital, and/or the like. Interrelated systems may use the exchanged data to operate, for example, in a surgical procedure, post-procedure analysis, maintenance determination, and/or the like.

Data degradation may occur in exchange of data and/or monitoring of surgical systems providing data. For example, a first surgical system may obtain data associated with a second surgical system (e.g., via monitoring the second surgical system). Monitoring the second surgical system may affect the data, for example, being generated, measured, etc. The data quality may be affected. For example, monitoring a device may affect the processing, latency, and/or sampling rate associated with data collection, generation, and/or measurement. The affected data may lead to inaccurate data. Inaccurate data may affect other systems that use and/or rely on the data being generated and/or collected.

Monitoring of data of a surgical system may be unwanted. A first surgical system may monitor a second surgical system for data. The first surgical system's monitoring may be performed covertly (e.g., without the second surgical system's knowledge) and/or transparently (e.g., with the second surgical system's knowledge). A surgical device (e.g., second surgical system) may not want to be monitored, for example, to preserve the integrity of the data collection and/or operation. The surgical device may not want to be monitored, for example, based on privacy concerns associated with the data collection and/or surgical system's operation. The surgical device may not want to be monitored, for example, by unauthorized access (e.g., only wants to be monitored from trusted sources).

Based on detection of being monitored, a surgical system may want to adapt to preserve data integrity and/or operation integrity (e.g., making sure the data is accurate and unaffected and/or operation/processing is accurate and unaffected). A first surgical system may determine that it is being monitored by a second surgical system. The first surgical system (e.g., surgical system being monitored) may determine (e.g., think) that the monitoring is affecting the data collection and/or first surgical system's operation. The first surgical system may want to adapt operating configurations (e.g., parameters) to avoid data issues (e.g., data inaccuracy) or operation issues (e.g., improper processing/operation). The first surgical system may employ techniques to prevent the monitoring from occurring and/or techniques to prevent the monitoring from affecting the first surgical system's operation and/or data collection.

The second surgical system (e.g., performing the monitoring of the first surgical system) may want to stay covert (e.g., outside of the first surgical system's awareness), for example, to monitor data based on an original operating configuration used by the first surgical system. The second surgical system may not want the first surgical system to change its operating configuration (e.g., parameters). The second surgical system may employ techniques to stay hidden from the first surgical system's detection of monitoring.

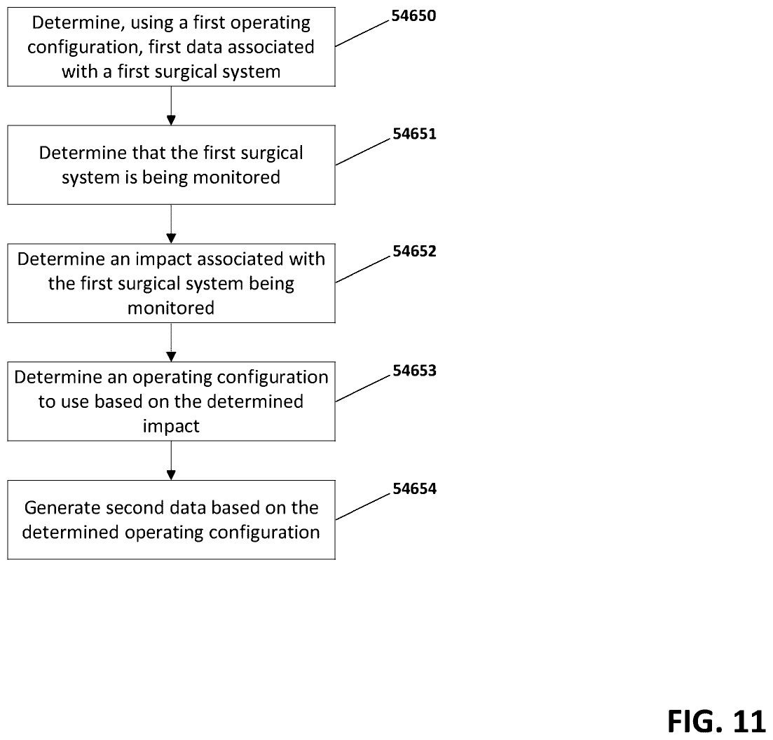

A first surgical system may adapt its operating configuration (e.g., parameters) to prevent monitoring (e.g., unwanted monitoring) of its operation and/or data. FIG. 11 illustrates an example flow of adapting operating configurations associated with data being monitored or received. The first surgical system may operate using a first operating configuration. As shown at 54650, first data associated with a first surgical system may be determined (e.g., using a first operating configuration).

A second surgical system may monitor the first surgical system. The monitoring of the first surgical system by the second surgical system may affect the data (e.g., data accuracy) generated by the first surgical system and/or the operation/processing of the first surgical system. The second system may be intercepting, copying, and/or viewing the data generated by the first surgical system.

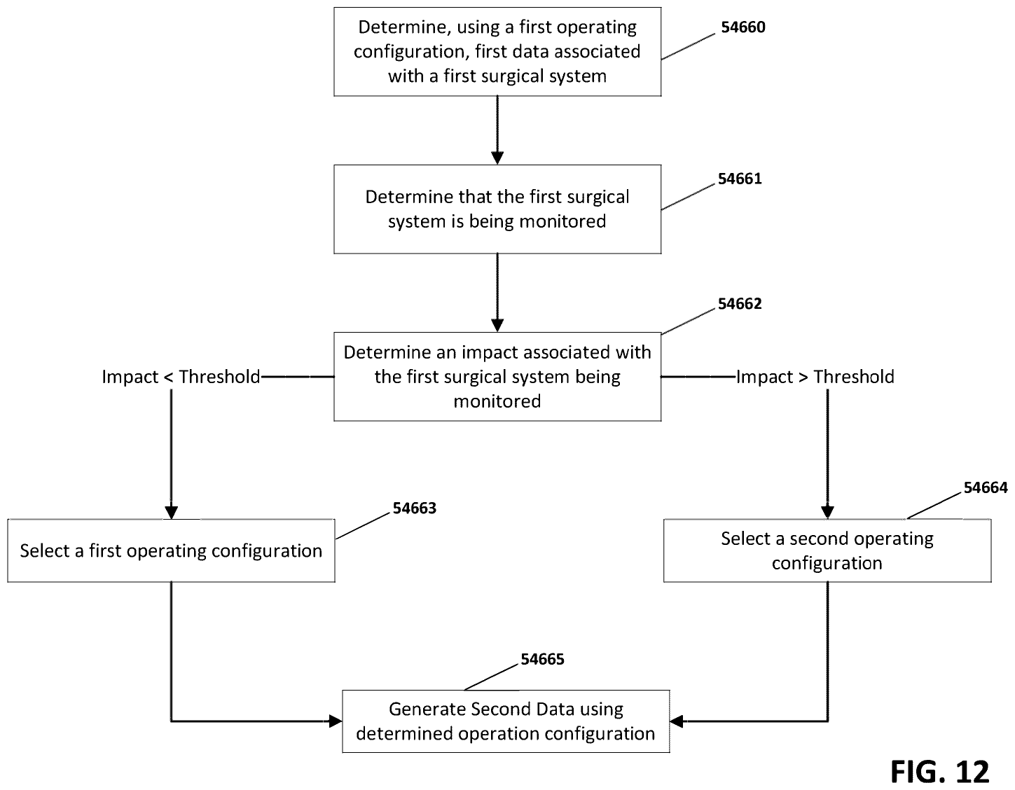

As shown at 54651, it may be determined that the first surgical system is being monitored. A first surgical system may determine that it is being monitored based on the data it is generating and/or an impact on its operation. For example, the first surgical system may determine a data degradation and/or signal loss. The degradation and/or signal loss may be attributable to being monitored. The first surgical system may determine that data quality is below a threshold (e.g., as described herein, with respect to FIG. 12). Based on the determination that a portion of the first surgical system's operation is being impacted, the first surgical system may determine that it is being monitored.

For example, an impact associated with the monitoring of the first surgical system may be determined (e.g., as shown at 54652 in FIG. 11). The impact may be associated with how the monitoring of the first surgical system is impacting the operation and/or data collection by the first surgical system. The impact may include an impact on the data collection. For example, the impact may be a difference in expected measurements in data as compared with actual recorded and/or sensed measurements. The impact may include a latency and/or delay in sensor measurements. The impact may include an impact on the operation and/or processing of the first surgical system. For example, the impact may include a delay or effect on operation speed.

The first surgical system may adapt its operating configuration (e.g., parameters) based on the determined impact. As shown at 54653, an operating configuration to use may be determined based on the determined impact. The operating configuration may be the same operation configuration (e.g., first operating configuration) being currently used, for example, if the impact is insignificant (e.g., below a threshold). For example, if the monitoring impact on the first surgical system is insignificant and/or negligible, the first surgical system may determine to maintain the current operating configuration. The first surgical system may determine to use a second operating configuration based on the impact (e.g., the impact being greater than a threshold). The second operating configuration may include a modified operating parameter. The second operating configuration (e.g., operating parameter) may be determined based on the determined impact.

The determined operating configuration may include an operating configuration that prevents the monitoring from occurring. The determined operating configuration may include an operating configuration that maintains the integrity of the first surgical system's operation while also allowing the monitoring to continue. For example, the monitoring may be allowed as long as the first surgical system can maintain data and/or operation integrity.

As shown in FIG. 11 at 54654, second data may be determined (e.g., measured, generated, sensed, etc.) based on the determined operating configuration. The second operating configuration may be used to perform processing and/or operation associated with the first surgical system.

FIG. 12 illustrates an example flow of adapting operating configurations associated with data being monitored or received with respect to an impact on the data. As described herein, an impact on operation and/or data collection may be determined for a surgical system being monitored. For example, a first surgical system may determine that a second surgical system monitoring the first surgical system impacts the operation and/or data collection of the first surgical system. The first surgical system may determine whether the impact is severe enough to warrant adapting an operating configuration to maintain the integrity of operation and/or data integrity.

As shown at 54660 in FIG. 12 first data may be determined (e.g., collected, received, sensed, measured, processed, etc.). The first data may be associated with the first surgical system. The first data may be associated with the first surgical system using a first operating configuration. It may be determined that the first surgical system is being monitored (e.g., as shown at 54661 and as described herein (e.g., with respect to FIG. A)). For example, it may be determined that the first surgical system is being monitored based on the first data (e.g., data is inaccurate and/or impacted).

An impact associated with the monitoring may be determined (e.g., as shown at 54662 in FIG. 12). The impact may be associated with a difference in expected data as compared with the actually collected and/or measured first data. In examples, first data may indicate that an EKG lead had a loss of signal at a point in time. The expected data associated with the first data may indicate that there is no EKG lead signal loss. Therefore, an impact may be determined based on comparing the expected data indicating that there is no signal loss at the point in time versus the actual first data indicating signal loss at the point in time. The impact may affect the first surgical system's operation and/or operation associated with other surgical systems that use the data generated from the first surgical system.

An impact associated with a magnitude of a signal may be determined. For example, the system may use a known signal or digital response to determine whether a magnitude of the signal within the line is being monitored (e.g., taped for another reading). For example, the magnitude may differ from the expected known magnitude if the surgical system is being monitored. The surgical system may introduce a known temporal based signal to a main signal and examine the return speed or transmission speed of a phase to determine whether the cables or connectors have a different speed than anticipated.

An impact associated with electronic properties of interconnections may be determined. The electrical properties of interconnections may be monitored and compared with a previous and/or known value, for example, to determine the transmissibility of the interconnections and any intercepts (e.g., monitoring by other devices).

For example, an echo cardiogram (ECG) sensor may use multiple leads (e.g., 3 to 7 leads). The ECG sensor may sense measurements. Interception of a small electrical signal from the ECG signal may lead to signal loss due to being monitored by one or more sources. The primary source may then interpret the inaccurate data (e.g., due to signal loss). The interpretation may be inaccurate (e.g., interpretation may indicate a physical flaw or abnormality that only exists because of the monitoring. The processing of the incorrect data may lead to an incorrect interpretation of a heart issue, which may cause a system to incorrectly operate or send incorrect notifications.

Transformed data may be impacted. For example, transformed data may be intercepted (e.g., based on a monitoring). The interception of the transformed data may introduce a latency in the signal. The latency of the signal, for example, may affect data (e.g., heartbeat shape), which may lead to inaccurate interpretation of the data.

A sensor or smart probe may be used to determine an impact, for example, to determine whether a signal is outside of an expected range. If the signal is determined to be outside the expected range, it may be determined there is an impact and that the system is being monitored.

Signal leads and/or power leads may be used to determine monitoring behavior. For example, a power lead may be used to monitor any additional draw associated with a monitoring system. If the system has the capacity to handle the additional load and the dip in voltage is within specifications, monitoring may be determined to be acceptable (e.g., from a power standpoint). Signal leads may be used to monitor signal lines for signal loss, phase shift, and/or impedance loss.

Impact may be determined based on mapping data streams. For example, mapping of data streams may indicate emitted pulses associated with the surgical systems. Based on the emitted pulses, changes in the emitted pulses may be determined. An impact may be determined based on the determined changes in emitted pulses.

An operating configuration to use may be determined based on the determined impact. A threshold associated with an impact based on monitored may be determined. The threshold may be preconfigured. The determined impact may be compared with the threshold to determine an operating configuration to use (e.g., as shown in FIG. 12).

A surgical system may use identification of monitoring to determine the impact of the monitoring. For example, the surgical system may determine an anticipated impact rating that is associated with the data being monitored. For example, the impact may be associated with a determination of an associated risk of the data being monitored. For example, patient health information, treatment information, business critical information, business intellectual property information, and/or general data may have respective risks of being monitored. At least based on the privacy requirements, risk associated with patient health information may be higher than other types of information, if it is monitored. Business critical information may be associated with data that may include network access keys to critical infrastructure. Business intellectual property information may include information associated with algorithms for operations of devices. General data may include information associated with power consumption levels of a device.

An inherent and/or detected risk associated with the monitoring may be determined. For example, interference with therapeutic functions and/or interference with business functions may be determined. Interference with therapeutic functions may be associated with a monitoring of an EKG lead which may lead to reduced accuracy of the lead (e.g., based on an impact of the electric signal associated with the monitoring). Interference of business functions may be associated with a monitoring of an intellectual property stream which may impact the quality of service of the network, which may reduce the ability of the network to function as intended.

An indirect determination of the impact of monitoring and/or sensing may be determined. A system being monitored may have partial or incomplete information to detect a total impact of the monitoring. The system may indirectly assess the monitoring and/or the impact on the operating system. For example, the system may indirectly assess the monitoring and/or impact based on coordination with other systems and/or known prior correlations. For example, a smart system may detect an anomaly in part of its operation, but may have limited insights into the anomaly. The smart system may alert a secondary system (e.g., surgical hub) which may confirm and/or provide insight on the anomaly. In examples, a system may flag an anomaly if a lead (e.g., new, unexpected lead) is connected to it. A system may flag an anomaly if the leads are incorrect and/or mis calibrated. After a number of occasions of altered leads being detected, the system may flag the error and indicate that the error is likely not because of the leads, but rather an unauthorized use of the system and/or leads.

A first surgical system may determine whether a second surgical system monitoring a data stream is attempting to observe, obtain, or edit data. For example, the second surgical system may be observing or obtaining data associated with device ID, manufacturing ID, device component and/or sub-assembly, calibration data, use and/or count cycle, electrical data (e.g., to check device functionality), patient data, and/or the like. The monitoring performed to observe or obtain data may be performed by surgical hubs and/or maintenance systems to determine maintenance information associated with surgical systems. For example, the second surgical system may be attempting to edit and/or adulterate data, such as, for example, updating calibration information, updating use/count cycle (e.g., device reprocessability), editing algorithm and/or operating specifications (e.g., optimizing device functionality), modifying patient data, and/or the like.

The surgical system may determine the importance associated with the data being monitored. For example, the surgical system may determine whether important data or unimportant data is being monitored. The surgical system may determine whether the system is in a closed loop (e.g., responds to isolated system). The surgical system may determine an amount of data being measured.

The surgical system being monitored may identify a (e.g., each) signal line that is being monitored. For example, the monitored system may monitor the monitored lines to ensure the output signal specifications stay within design requirements of the original signal. If the monitored lines fall outside specified levels, the surgical system may perform one or more of the following: halt signals to minimize hazards that a lower level signal may create, increase output signal to compensate for loss to outside of specified levels, alert for the potential of corrupted signals, and/or the like. The monitored lines information may be collected into a data set for future analysis (e.g., to prevent unforeseen issues in the future).

The surgical system may react to being monitored. For example, the surgical system may select an operating configuration based on the fact that it is being monitored.

In examples a first operating configuration may be selected (e.g., determined) based on the impact. As shown in FIG. 12 at 54663, the first operating configuration may be selected. The first operating configuration may be selected based on the determined impact being less than the threshold. For example, it may be determined that the monitoring has an insignificant impact, and therefore it may be determined to maintain the current operating configuration (e.g., first operating configuration) being used.

In examples, a second operating configuration may be selected (e.g., determined) based on the impact. As shown in FIG. 12 at 54664, the second operating configuration may be selected. The second operating configuration may be selected based on the determined impact being greater than the threshold. For example, it may be determined that the monitoring has an impact (e.g., greater than a threshold) that is negatively affecting operation and/or data collection. Based on determining that an impact is negatively affecting operation and/or data collection, a second operating configuration (e.g., operating configuration parameter) may be determined. The second operating configuration may be predefined and/or determined based on the impact.

In examples, the surgical system may alter functional operations (e.g., reduce functionality) to minimize the impact of the monitoring/surveillance on its operations. For example, a smart system may minimize a number of operating system files, modules, sub-programs being used. This may prevent the monitoring system from impacting the inherent operating of the smart system. For example, the surgical system may alter functional operations by performing one or more of the following: change access or levels of activity; increased protections and/or mitigation; cease of further transmission; denial of audible transmission; video denial; system shutdown; system lockout’ multiplexed data signals; etc.

The surgical system may change access or levels of activity. For example, the change in access or levels of activity may depend on a surgical procedure. For example, it may depend on a surgical procedure step risk level and/or a stage of the surgical procedure. The surgical system may reduce available services, such as, for example, refraining from performing functions, if/when the surgical determines that it is being monitored. For example, a surgical procedure may be planned. Before the surgical procedure begins, the surgical system may determine and/or detect that it is being monitored (e.g., by an unknown entity). The system may disable its communication and alert a user (e.g., an HCP) that it is refraining from functioning, for example, based on the determination and/or detection that it is being monitored. For example, a surgeon may be preparing to fire an endocutter across a critical structure. At the same moment, a surgical system may detect that it is being monitored by an unknown entity. The surgical system may determine (e.g., based on the detected monitoring) a risk of ceasing operation associated with the firing of the endocutter. The surgical system may determine the risk of ceasing the firing of the endocutter is too impactful (e.g., would lead to patient risk). The surgical system may determine to continue operation despite the monitoring, for example, because the determined risk is high (e.g., higher than a threshold).

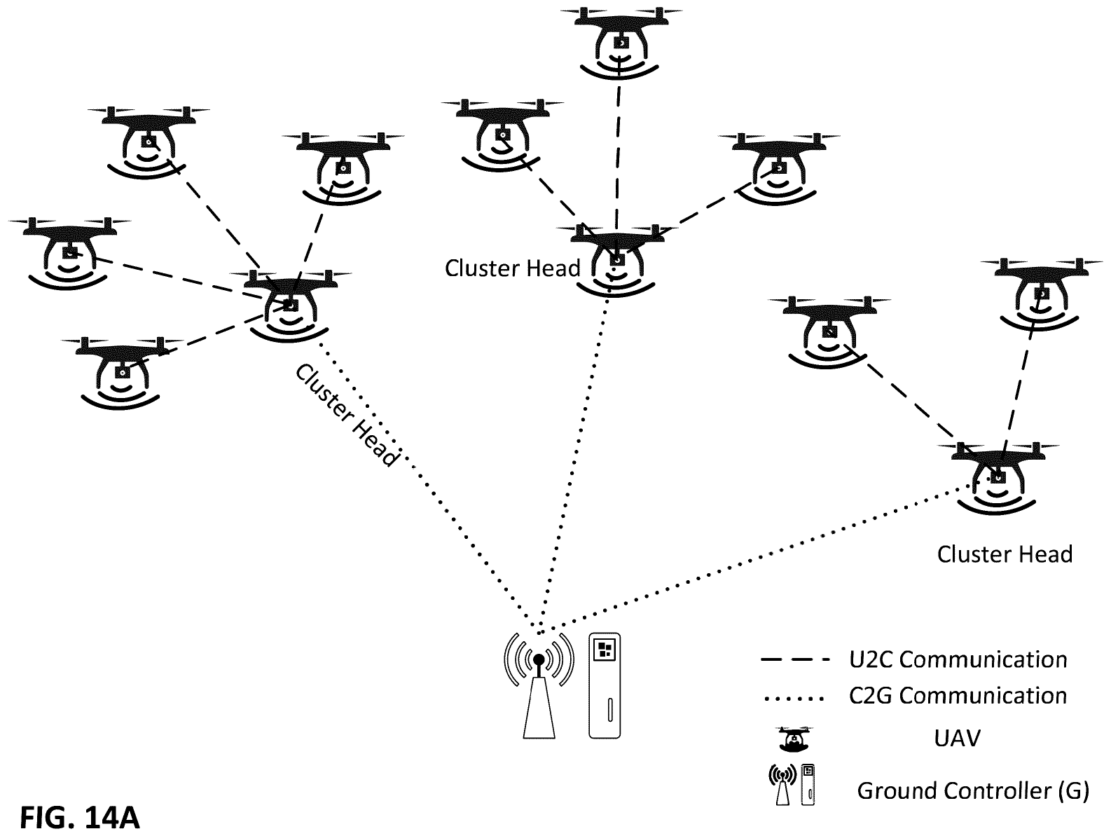

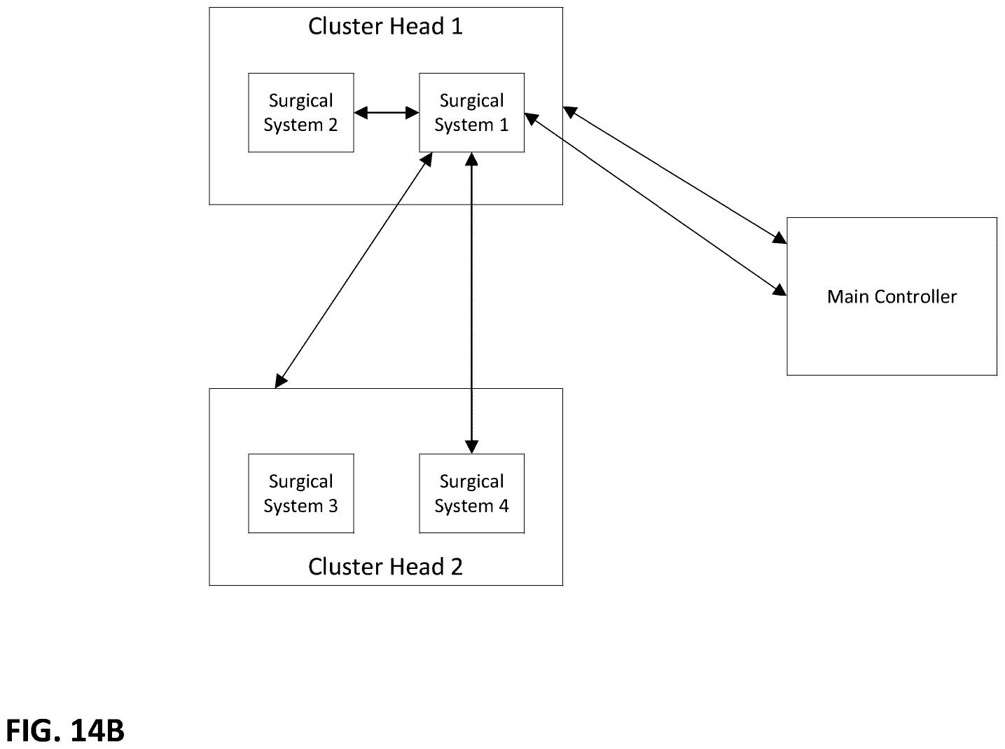

The surgical system may increase protections or mitigate monitoring in response to a detected monitoring. For example, key strength adjustments may be used. FIG. 14A illustrates an example of a system adapting operating parameters based on a detection that the system is being monitored. A system may determine that a potential adversary is monitoring the transmission of messages. The system may determine to adapt one its operating parameters or features (e.g., encryption keys that are used for coding the communication channels between various devices of the system). For example, the system may change one of the operating parameters or features (e.g., the encryption keys from 128-bits to 256-bits). In an example, the system may make the change for increased protection. The increase may result in increased computational costs, but it may also increase the complexity thereby resulting in reduction of the unwanted monitoring. As shown in FIG. 14A, communication may be exchanged between cluster heads, cluster heads and the ground controller, and/or between cluster heads and the other devices of a cluster. Any of these communication may be altered, for example, based on a detected monitoring. FIG. 14B illustrates an example of communication exchanged between cluster heads, the main controller, and/or devices of a cluster. As shown in FIG. 14B, a surgical system 1 may communicate with one or more of surgical system 1 (e.g., device within the same cluster, Cluster Head 1), the cluster head 1, the main controller, a different cluster head (e.g., cluster head 2 as shown in FIG. X), surgical system 4 (e.g., a device of cluster head 2), and/or the like. For example, surgical system 1 may determine to change an operating configuration. Surgical system 1 may communicate the change of operating configuration to the other devices in the same cluster head, devices of other cluster heads, the main controller, and/or the like. The changes in the operating parameters associated with any of those communications may prevent unwanted monitoring. Other adaptable operating parameters may include dynamic key adjustments, dynamic cyclic redundancy check (CRC) adjustments, changing the encryption protocol (e.g., from a static to a dynamic encryption type) and/or the authentication mechanism between the devices to provide robust communication channels that are less susceptible to monitoring.

The surgical system may cease further transmission, for example, in response to a detected monitoring. The system may halt transmitting further data along a determined section (e.g., compromised section).

The surgical system may deny audible transmission in response to a detected monitoring. For example, an audible jamming tone may be created and/or used. The audible jamming tone may be outside the audible region for staff but within a frequency response range of audible equipment. The audio output may be adjusted. White noise may be inserted. The white noise may target specific frequency ranges.

The surgical system may deny video. For example, a non-visible light source that may be captured within a screen may be used. The non-visible light source may blind a video source.

The surgical system may perform a system shutdown in response to a detected monitoring. The system may delete data from itself. The system may corrupt internal memory, for example, to deny access to its data. For example, repeated attempts to access portions of the system (e.g., that do not feature a clinical aspect, such as, removing a panel that provides access to a circuit card assembly) may result in the circuit card assembly wiping out readable memory so it may not be accessed.

The surgical system may perform a system lockout, for example, in response to a detected monitoring. The system may lock out access to the system for a period of time. The lockout may be in response to repeated unsuccessful access attempted. The system may lock out access to the system based on how the system is being accessed. For example, a system may detect that panels related to the system for accessing a microcontroller assembly (e.g., which is usually only performed during maintenance servicing) or may detect that a service port is actively in use. In response, the system may shut down operation of unrelated portions of the system, for example, as a safety precaution and/or to limit the ability to monitor the system.

The surgical system may multiplex data signals, for example, in response to a detected monitoring. A carrier wave (e.g., larger carrier wave) may be used for transmission of data. The data may be embedded onto frequencies on the larger carrier wave. In examples, an AC power line may include digital information and may serve a purpose of providing data to a system (e.g., in addition to powering transmission of the data).

The surgical system may disable external access points that may not be part of the data collection segment(s). For example, the surgical system may remove accessibility of universal serial buses (USBs), output ports, video outputs and/or audio outputs to external monitors, turning off wireless receivers, and/or the like. The smart system may shift network connections to an alternative, separately secured network (e.g., virtual private network (VPN), etc.).

The surgical system may use offensive counter responses to a determination that it is being actively monitored. For example, the system's counter response may attack the system that is performing the monitoring. For example, an offensive counter response may include one or more of the following: denial of wireless transmission; intentional transmission of incorrect information; noise generation; cybersecurity data attack (e.g., embed attack vectors within data being transmitted); methods of prevention; denial of power; inclusion of a parameter into a control loop to introduce monitoring disturbance, and/or the like.

The surgical system may perform denial of wireless transmission as an offensive counter response. The denial of wireless transmission may include frequency jamming. For example, the system may perform wideband frequency jamming. For wideband frequency jamming, the system may attempt to wirelessly spam available frequencies, for example, to halt data transmission along those frequency channels. For example, the system may perform selective frequency jamming. The system may attempt to wirelessly spam selective frequencies to halt data transmission along the frequency channels.

The surgical system may perform intentional transmission of incorrect information as an offensive counter response. The system may intentionally embed false and/or misleading information within surgical data that may be sent or copied by the monitoring system.

The surgical system may perform noise generation as an offensive counter response. For example, the surgical system may generate an acoustic pulse. The acoustic pulse may be generated within the audible range or outside the audible range. The system may actively generate noise within or external to the audible range, for example, to block signals or drown out relevant information from the other system attempting to monitor or collect it. In examples, the system may become aware that a secondary system is monitoring it. As a result, the system may emit a high frequency signal at random intervals. The high frequency signal may fall outside the audible range, for example, to avoid bothering staff or patients. The high frequency signal may be within the frequency response range of microphones. Therefore, the random intervals of noise may block out signals that may be difficult to filter out.

The surgical system may generate light noise. For example, light source noise may be interfered with using an infra-red light. The surgical system may detect that someone is filming the system. The system may enable an infra-red light source that may not be human detectable. The infra-red light source may impact the quality of recordings.

The surgical system may adopt methods of prevention, such as, for example, changes in communication method, time based actions (e.g., intentionally swapping data across lines at predefined random intervals, for example, where a data pathway may indicate to other lines which data pathways may switch), using multiple data lines, movement of the system, changes in signal voltage, differential signal utilization (e.g., using differential signals to require a competing signal to actively monitor multiple lines to monitor a single data stream), timestamping of data (e.g., rejecting data as falsified if data is not received during a period of specified time), and/or the like. For example, communication methods may include overloading lines to allow multiple communication methods. The surgical system may switch from a first communication (e.g., UART to ModBus) to another, for example, to help prevent the ability of the monitoring system from reading data. Multiple data lines may be used, for example, such as intentionally redundant data lines which may allow switchover in response to data monitoring. Cryptographic splitting may be used. Cryptographic splitting may include intentional transmitting of half of an encrypted message on a first channel and a second half of the encrypted message on a second channel. The receiving system may recompose the message from both data streams. Movement of the system may be used to alert the system that it is being monitored (e.g., detection of physical movements that do not intrinsically make sense. For example, a piece of capital equipment may detect (e.g., using an accelerometer) that it is being moved during a surgical operation. The movement may be analyzed to determine whether monitoring of the equipment is occurring. For example, physical movement of the system may include removing of installed panels. Changes in signal voltage may be used to prevent monitoring. For example, voltage on a signal may be reduced, for example, to lower a signal to noise ratio to reduce the ability of competing equipment to effectively monitor the system without using dedicated equipment. Bandwidth detection and/or monitoring of data transmission physical parameters (e.g., voltage and current of transmitted data) may be monitored.

The surgical system may use denial of power as an offensive counterattack on being monitored. The system may actively deny power to competing systems, for example, so the competing systems do not function. The system may notify OR staff and may request to remove power from a monitoring device. The system may turn off power not a bank of circuits, for example, via a smart power strip.

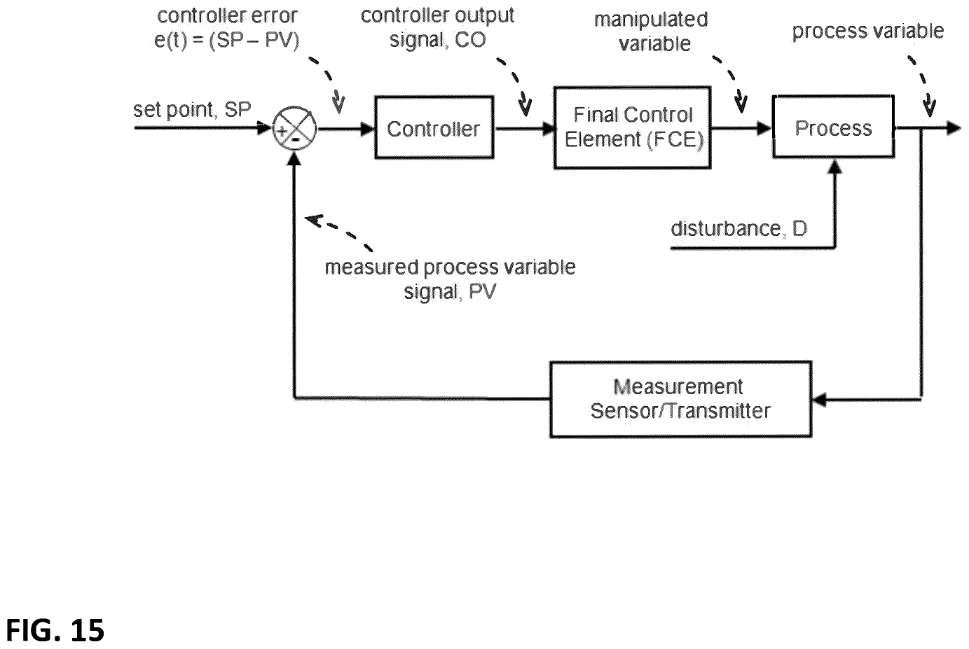

The surgical system may comprise a process control to monitor certain conditions or measurements that may need to be corrected. The surgical system may include a known monitoring disturbance in such process control and observe reaction of the surgical system's (e.g., the controller of the surgical system) response to the included or injected monitoring disturbance. FIG. 15 illustrates an example of a system adapting operating parameters to disturb a detected monitoring. As illustrated in FIG. 15, a set point or a target value may be defined for a process variable associated with a process. When all conditions of the process are satisfied, the process variable is equal to the setpoint. A disturbance, D may be injected or included in the process that is being monitored. The disturbance may alter the system operation such that some corrective action is required. Once the measurement sensor measures the disturbance, the controller of the control loop may receive a non-zero error signal e(t)=(Set point value, SP—Measured process variable signal, PV). The controller may generate an output to activate the final control element (FCE), which may be an electronic or a mechanical system used to control the manipulated variable. The manipulated variable may control the process variable, which may be the controlled variable part of the process that is being controlled. The process may be controlled until the value of the setpoint is again the same as the process variable.

The surgical system may determine minimum operating parameters for device operation (e.g., for operation in safe mode). For example, minimum operating parameters may include one or more of the following: reverting to manufacturing default; excluding non-original equipment manufacturer (OEM) sensor inputs; airplane mode (e.g., disabling features but preserving laparoscopic video, and/or system switching off everything streaming); eliminating everything except video (e.g., all data displays turn gray); blank out EKG and show heart rate (e.g., only heart rate); and/or the like.

As shown at 54654, second data may be generated based on the selected (e.g., determined operating configuration). The second data may be exchanged with other surgical systems within the surgical environment.

In examples, a surgical system may determine that it is being monitored and determine an operating configuration to use based on the monitoring. For example, first data associated with a first surgical system using a first operating configuration may be determined. The first surgical system may determine that it is being monitored based on the first data. The first data may be impacted based on the first surgical system being monitored. The first surgical system may determine an impact (e.g., impact value) associated with the first surgical system being monitored. The first surgical system may determine (e.g., select) an operating configuration to use based on the impact associated with the first surgical system being monitored. The operating configuration determined may be the first operating configuration or a second operating configuration. The operating configuration may be determined based on the impact being compared with a threshold (e.g., select the first operating configuration if the impact is less than the threshold and select the second operating configuration if the impact is greater than the threshold). The operating configuration may be determined based on the impact being outside a threshold range (e.g., range associated with proper operation of the surgical system). The operating configuration may be determined based on the impact being greater than a threshold associated with the first data being inaccurate and/or incomplete data. The impact of the surgical system being monitored may be determined based on one or more of the following: a type of data associated with the first data; a risk associated with operating using the impacted data; a type of monitoring that is being used to monitor the surgical system; an importance associated with the first data; etc. The monitoring may be associated with observing data and/or editing the data. Based on the selected operating configuration, second data may be generated and/or determined.

In examples, the operating configuration selected based on the determined impact may be associated with preventing the monitoring. For example, the operating configuration may be associated with a safe mode parameter for operating. The safe mode parameter for operating may be associated with a reduction in functionality. The operating configuration selected based on the determined impact may be associated with a selective intervention operating configuration. For example, based on a determination that the impact is greater than the threshold, the determined operating configuration parameter may be associated with reducing collection of protected data and/or ensuring an integrity associated with generated data. The operating configuration selected based on the determined impact may be associated with a transform parameter, wherein second data is generated based on the transform parameter.

A first surgical system may desire to monitor a second surgical system covertly (e.g., without the second surgical system knowing that it is being monitored). For example, the first surgical system may want to covertly monitor a second surgical system so the second surgical system does not change its operating configuration. For example, the first surgical system may want to intercept data associated with the second surgical system without the second surgical system preventing access to the monitoring and/or operating with reduced functionality.

A system being monitored may request ceasing and desisting of intercepting, monitoring, and/or recording of a signal. The system may request cease and desist for parts of data stream that it recognizes as being impacted by monitoring. For example, the system may identify portions of the observation that may be inhibiting, affecting, interfering, or adulterating, and notify the observing system of the needs for the system to stop what it is doing (e.g., because it is negatively affecting the system being monitored). The monitoring may be diminishing the signal received by a main system, or noise or error may be introduced in the signal which may impact an ability to maintain proper resolution and/or speed. Impacts that may be relevant to a monitored data stream may include one or more of the following: changing the balance of the noise/signal ratio; introduction of latency; alteration of calibrated expected magnitude or ratio of signals; adulteration of headers or packet identifiers; impact on phase of signal oscillation; signal interference from recording system; creation of electro-magnetic fields that introduce error into a shielded system; drifting the relative ground to which the signal is measured from; bleed-over of the monitor signal back into the primary line through capacitive coupling or other electrical coupling means which could cancel out, reinforce, or additively or substantively impact a primary signal.

A system being monitored may communicate with surrounding systems (e.g., smart systems in the surgical environment or other devices or people in the surgical environment). The communication may include a warning and/or heads up to enable other smart systems to proactively prepare for monitoring. Proactive preparation may include enabling defensive protocols, such as, for example, using encryption codes and/or keys. For example, a visualization system may detect an intrusive system monitoring it, which may be physically attached to the system. The visualization system may notify a separate smart system that it has been compromised.

To covertly monitor a second surgical system, the first surgical system may adapt its operating configuration to avoid detection. For example, the first surgical system may use an operating configuration associated with minimizing an impact on the second surgical system's operating and/or data collection.

A surgical system may transform data based on a determination that it is being monitored. The transformation of the data may be performed to confuse the monitoring system. For example, the data streams may be transformed. The coordinate system (e.g., coordinate transformations) may be transformed. For example, the coordinate system information may include a number line, cartesian coordinate system, polar coordinate system, cylindrical and/or spherical coordinate system, homogenous coordinate system, curvilinear coordinates, log-polar coordinate system; Plucker coordinate system; generalized coordinates; canonical coordinates; barycentric coordinate system; trilinear coordinate system; and/or the like. The surgical system may transform data between the various coordinate systems to confuse a monitoring system.

Adaptive behavior of a surgical system may be used based on monitoring by another system that may not be stopped. For example, the system may determine portions of the control system that may be affected by monitoring and may remove that portion from a closed loop control aspect of the affect system control. Identification of sensor feeds that are adulterated, impacts, or no longer reliable due to external monitoring and/or indicating that feed as unreliable or requiring supplementary conformation may be performed. A closed loop control signal may be replaced with an open loop with a predefined parameter. Exchanging an effective closed loop control signal with an alternative signal (e.g., that is not monitored or not affected by monitoring) may be used. Additional oversight monitoring or constraining adjustable parameters of a closed loop system may be applied, for example, to ensure affected control loop adulterated sensor feeds do not have a large impact on the operation of a closed loop operating system.

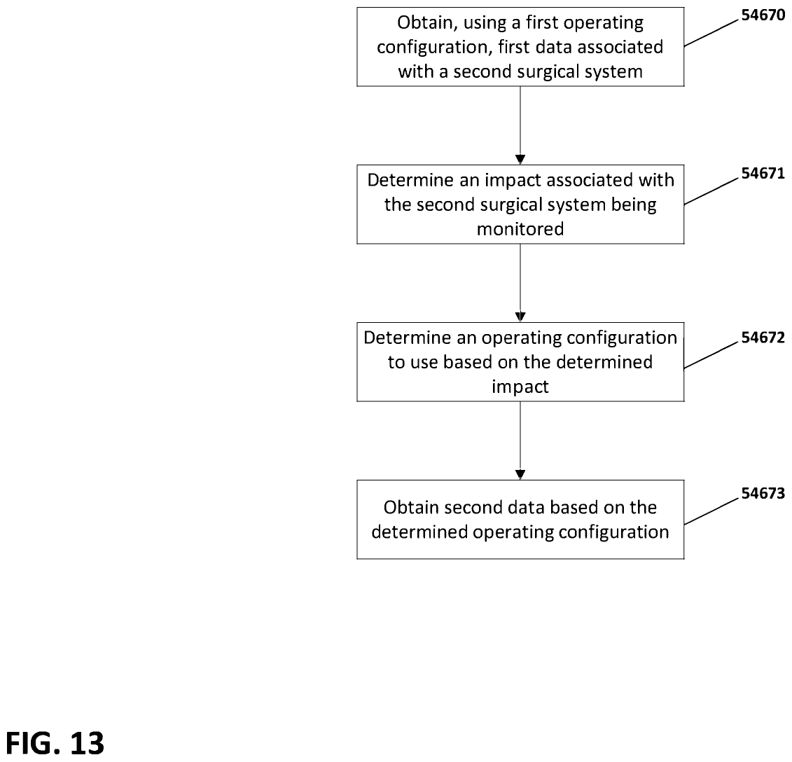

FIG. 13 illustrates an example flow of a surgical system changing its operating configuration to avoid being detected that it is monitoring a different surgical system. A first surgical system may monitor a second surgical system. As shown at 54670, first data associated with a second surgical system using a first operating configuration may be obtained (e.g., by a first surgical system).

As shown at 54671, an impact associated with the second surgical system being monitored may be determined. For example, the first surgical system may determine whether its monitoring of the second surgical system is impacting the operation and/or data collection of the second surgical system. The impact may be determined as described herein with respect to FIG. 12. The first surgical system may want to minimize the impact on the operation of the second surgical system, for example, so it remains undetected. If the impact on the operation of the second surgical system is noticeable, then the second surgical system may adapt its operating configuration to prevent the first surgical system from monitoring the second surgical system.

As shown at 54672, an operating configuration to use for the first surgical system may be determined, for example, based on the determined impact. The first surgical system may select a second operating configuration (e.g., current operating configuration) for example, if the determined impact is below a threshold (e.g., the second surgical system would not notice the impact and/or the impact is negligible).

The first surgical system may select a third operating configuration, for example, if the determined impact is above the threshold. The third operating configuration may include a parameter that is adapted to avoid detection from the second monitoring system.

As shown at 54673, second data may be obtained from the second surgical system using the determined operating configuration associated with the first surgical system. The first surgical system may continue to monitor and/or intercept data from the second surgical system using the determined operating configuration.

A first surgical system may adapt its operating configurations to covertly monitor a second surgical system and avoid detection of the monitoring. The first surgical system may obtain (e.g., using a first operating configuration) first data associated with a second surgical system. The first surgical system may determine an impact associated with the second surgical system being monitored, for example, based on the first data. The impact may be associated with inaccurate data and/or degraded data. The first surgical system may determine an operating configuration to use (e.g., maintain the first operating configuration or determine a second operating configuration) based on the determined impact. The operating configuration may be determined based on comparing the determined impact to a threshold (e.g., range of values). The determined operating configuration may include using an operating parameter. The operating parameter may be associated with an impedance. For example, a first operating configuration may be associated with using a first impedance and the second operating configuration may be associated with using a second impedance. The operating configuration may be determined, for example, based on a type of data or a level (e.g., privacy level) associated with the data. The first surgical system may continue to monitor the second surgical system and/or intercept data associated with the second surgical system. Second data associated with the second surgical system may be obtained, for example, using the determined operating configuration.

In examples, a surgical system may monitor another smart system using a predefined means to export the data from one system to another. The data export may be a unidirectional stream. The data export may not be monitored by the first system to determine whether systems are watching the output.

For example, data export streaming may be used for external use or monitoring. A first smart system may have a data output port that streams data out for other systems to use. The first smart system may not monitor or care if the data is monitored or not. In examples, an EKG output of a heart rate monitor may be connected to other systems, for example, allowing them to monitor the feed provided by the system (e.g., without interfering with the system's monitoring and display of patient heart rate). The output may be ported into the imaging system of the catheter ablation system and/or displayed on the screen adjacent to a live image (e.g., allowing the HCP to see the heartbeat irregularities visually as well as a neuro component).

For example, data export streaming may be used for oversight. A smart system may be designed to be a first portion of a cooperative biomarker monitor, a leading indicator to a second monitored system, and/or a conformational means of the second control system. A raw or transformed data stream may be provided from the system with no feedback to a receiving system. The receiving system may use the provided secondary stream to confirm or ensure that its measure or monitored aspect is correct, the signal did not drift, and/or the stream is not out of sync with real-time aspects of the procedure.

In examples, a heartbeat may be measured by a blood pressure cuff. The blood pressure cuff may be capable of measuring the pressure within the extremity to which it is attached. The blood pressure cuff may include a built in beat monitor used to determine if pressure is exceeded and when it re-establishes. Automated cuffs may have an orientation issue if placed on the extremity (e.g., the internal sensor may need to be aligned to the artery of the extremity, such as, for example, the tube of the automated cuffs may need to be in the hinge of the elbow and the cuff may need to be an inch above the elbow). In surgery, a patient may be draped off to HCPs may no longer see the cuff or extremity as they are on the other side of the drape. The patient may be instrumented with EKG leads and a pulse oximeter (e.g., other smart systems that monitor heartbeat rate and timing). If the EKG leads and pulse oximeter supply the beat cadence to the blood pressure cuff it may differentiate between erroneous measurements of its monitor of blood flow from irregularities of the heartbeat itself.

Surgical systems may synchronize smart system outputs. An output stream from a first smart system may be provided to input into a second smart system, for example, to ensure that the two signals are synchronized and the use of the data together is ensured to be in sync. A third smart system may export a stream that two cooperative monitors use, or the third smart system may use a stream from one cooperative system to synchronize a second stream. In examples, an EKG monitoring system and a flexible scope catheter view may be used in cooperation during a surgical procedure (e.g., AFIB focal ablation). In between heartbeats, the catheter RF electrode may be advanced and single point ablation may be performed, and retracted before a subsequent heartbeat. The catheter control and imaging may be performed within the same smart system, but there may be a lag between detection and display of the video feed. An external heartbeat measure or EKG may be used to synchronize the systems. The EKG (e.g., used to determine whether ablation sufficiently stopped the irregular beat) may determine whether follow on point ablations are necessary. Both systems may benefit from ensuring synchronization of the signals to real-time patient heartbeats.

Surgical systems may listen to each other, for example, other systems within an audible range. An audible pulse of a first system may be used to alert a second system. The audible pulse may indicate an action to be taken. The audible pulse may indicate an error has occurred. For example, an audible emission may be generated and emitted, for example, to indicate that an endocutter is about to be fired.

Surgical systems may perform visual observation of other systems. The visual observations may include a light source. A light emitted diode (LED) alignment system may be used to enable visual observation of systems.

Signal surveillance of data streams via interception of extractions of external systems may be performed. For example, intercepted monitoring may include where a data stream is intercepted, copied, and parallel transmitted. The signal may be extracted from physical properties of a primary system's electrical or mechanical system. A second signal beside the first system primary use may be generated, for example, without the knowledge and/or cooperation of the first system. The second signal may be monitored and/or used to control a closed loop aspect of a second smart system.

For example, multi biomarker smart monitoring systems (e.g., CareOne, which may monitor Po2, CO2, ECG, temperature, blood pressure, etc., and a ventilator), may intercept return feeds or display outputs to obtain data. A system monitoring a data stream may show a graph, for example, based on the data streams. For example, a ventilator may intercept data from a CareOne device and show a graph representing a tidal volume changing to balance bloodgases CO2 from CareOne with exhalation gas CO2 from the ventilator in a closed loop manner.

A portion of a monitoring feed or related aspect of a smart system may be tapped into, for example, to enable simultaneous monitoring of a data feed by multiple systems or simultaneous monitoring of systems monitoring each other. For example, non-smart smoke evacuators may use a current sensor buckle that may be placed of a wire of an RF monopolar system wire that may detect the presence or absence of current in the cable (e.g., to know whether the generator is activated). The non-smart smoke evacuator may turn on when a current is detected. The turning on when a current is detected may be a form of covert monitoring. A smart smoke system may react to different intensities of activation with different velocities of motor speed. A smart smoke system may consider combined frequency and intensity of activation, for example, to adjust magnitude of smoke evacuation.

Data streams and/or a series of data points that have been intercepted, copied, and resent may be synchronized to other signals. Timestamping and/or packet ordering of data may be used to synchronize data. Intentional delay may be introduced, for example, to synchronize data. Dynamic delays in hardware or software (e.g., which may allow time to copy data and transmit) may be used. Dynamic delays may include speeding up slower pathways and slowing down faster pathways for better synchronization. Estimation theory may be used to create an illusion that a system is monitoring faster than it actually is. Mathematical synchronization may be used. Prior knowledge of system latency may be considered. Accommodation of the latency may be used to determine an optimized injection point. For example, a wirelessly enabled monitor may receive (e.g., need to received) messages within 50 mS of transmission). A system may artificially timestamp data to show it has been there in the correct order. A system may bypass other parts of the system where actions may be performed faster so the monitor can receive the data within 50 mS.

Signal loss and/or degradation minimization due to monitoring (e.g., surreptitious surveillance) may be mitigated and/or minimized. For example, fingerprints of surveillance may be removed to avoid detection of monitoring. In examples, temporal sampling, throughput, or speed of signal transmit may impact minimization. Phase matching and/or periodic spatial modulation of a coefficient may be used to minimize signal loss and/or degradation.

Dynamic impedance matching may be used to minimize an effect on a data signal from surveillance. For example, signal loss, impedance mismatch, or resistive change to the data stream may be monitored by intercepting or measuring the signal over time. A correction factor may be applied to the line impedance, resistance, or a signal boost, for example, to adjust for losses and/or changes induced by taking the measure itself or attaching the sensing system in-line with the signal. Dynamic impedance changes may be used. The system may use the impedance of the system as a flag to indicate if something has been attached to the system and may be monitoring it. A block box interception system may be used in-series with a signal and measure an expected impedance of the line before its introduction. The system may use parallel resistors to simulate the expected impedance in the parallel circuit to trick the independent system into thinking the black box does not exist in the system (e.g., to avoid detection).

Coupling of signals may be used to minimize an effect on a data signal from surveillance.

A microstrip may be used to electromagnetically couple a signal without physically interacting with it. A high speed data line may be monitored similar to an AC line. Overlaying of digital communication stream over an AC signal may be performed.

A man-in-the-middle technique may be used to minimize an effect on a data signal from surveillance. A monitoring circuit may be inserted to mimic an input and/output impedance of a larger circuit and may mimic associated amplitudes or other properties/characteristics. A power booster may be used to maintain signal level integrity so the signal comes out as it went in.

High impedance monitoring may be used to minimize an effect on a data signal from surveillance. A high impedance circuit may be used to monitor a signal with a minimal possibility of being monitored.

Type of data and/or level of data may be considered in determining how to monitor a data stream. For example, monitoring of therapeutic signals may use different monitoring techniques as compared with monitoring data signals. Risk based assessments may be performed for monitoring therapeutic data streams. Monitoring may change and/or adapt based on the type of data coming through. For example, if low energy sensing pulse from a generator is received, a different monitoring technique may be used from a data signal coming from a main therapeutic signal. A system may be interrogated to determine a monitoring technique. A change in monitoring technique may be used based on priority system information (e.g., system specification information, system data information).

Primary signal compensation may be used to minimize an effect on a data signal from surveillance. A primary signal may be compensated to accommodate that a monitoring signal may impact it. The monitoring network may acknowledge that it may impact the data, but may compensate the original signal so original characteristics are maintained. For example, a high frequency signal may have a known voltage peak, slew rate, and current draw (e.g., functions of its matched circuit). A monitoring circuit may be highly inducive, for example, due to additional wiring that is used to monitor it. A compensating circuit may be additionally installed to have more capacity and boost the signal to mimic the signal in addition to the monitoring circuit.

A data signal from a sensor to an amplifier and/or circuit board may be measured without impacting the signal strength or measure. Remote monitoring by an independent external source may be used. A data signal may be measured in line and a technique to resupply the data signal using transformers may be used.

An external supplemental monitoring system may be used to monitor capital box operation. For example, a smart power distribution system strip and/or surge protector may be used to determine which device is which (e.g., manually determine). Power may be used from the wall to check magnitude and plugs and tones may be used to determine which magnitude goes to which device. External monitoring systems may use secondary sensors to determine what it wants to measure (e.g., smart pad under a generator).

A smart device may be operated outside a larger communication network (e.g., may not be coupled to the larger communication network). The smart device external to the larger communication network may be monitored. For example, a smart network may have fuse level control over a power supply of the external device. Smart safety control of a Cone Beam CT may be enabled based on sensing, equipment, and/or location of HCPs. Localized communication networks may be interceded. For example, a system may use a denial of service style mitigation, for example, such as, for example, occupying existing local Bluetooth advertising channels, which may prevent a device from successfully pairing with another device.

An external system (e.g., uncontrolled system) may have impact on a smart system within the smart network. For example, the smart network may be adjusted to compensated for an uncontrolled system's action. The smart network may be adjusted, for example, to avoid unintended interaction. For example, an advanced monopolar energy generator that may not include an output monitoring port but whose activation may interfere with a patient biomarker monitoring system (e.g., EKG, pulse rate, PO2) may be used. The smart network may be adjusted to compensate for the uncontrolled system's action. For example, a smart system may control a smart AC distribution panel. The AC system may not be able to control what is plugged into it, but it may coordinate with controlled elements to mitigate risks, for example, such as dynamically enabling or disabling AC power ports to prevent tripping an entire breaker.

Comprehensive data may be collected. Expansion of the collected comprehensive data may occur from monitoring systems to determine the comprehensive data. Aggregation of monitored and communicated data may be performed to determine efficiencies.

Covert monitoring and/or surveillance may be non-intrusive. For example, a system may monitor actions associated with a surgery robot. The system may determine wear, maintenance intervals and/or the like based on the action, correction, or recalibrating that is used. The monitoring for the usage may affect the performance of the actions during surgery. The monitoring of the usage data may be performed at the end of the surgery, for example, to avoid impact on the actions.

Device operation may be adapted, for example, based on monitored patient biomarker data. Biomarker patient monitoring may enable identification of physiologic reactions that may affect smart device operation. For example, peripheral blood flow dilation may be linked to hypothermia, which may affect whether a patient heating system is used (e.g., based on monitoring the hypothermic indicating data). Controlled rate of change of the closed loop to adapt the magnitude of heat transfer may be used.

For example, blood sugar, heart rate variability, blood pressure, and/or the like may be monitored to determine implications associated with metabolic load or blood flow rate. The monitored information may be used to influence devices associated with drug uptake, core temperature generation, blood or tissue oxygenation, sedation dosage and/or depth, and/or the like.

For example, externally applied physiologic adaptation may drive a physiologic reaction that may drive a desired outcome. Use of therapeutic hypothermia to reduce the body's need for oxygen and changing the ratio of oxygenated blood to “critical” organs to re-balance CO2 based on O2-CO2 patient biomarker monitoring and mechanical ventilation constraints may be performed. Localized organ cooling may be used to induce localized therapeutic hypothermia to minimize ischemic tissue damage and reduce local bleeding relative to surgical intervention.

A first system may monitor a second system by monitoring the display of the second system. The first system may use a camera to monitor the room or display screen of the second system. The first system, based on the monitoring, may determine power level, activation, times, and/or the like. The monitoring via the display screen may not impact or be detected by the second system. For example, an electrosurgical generator may be monitored to identify which type of energy (e.g., bipolar, monopolar, etc.) is in use, power level settings, cut modes, coagulation modes, number of firings, etc., for example, which may be displayed on the display screen.

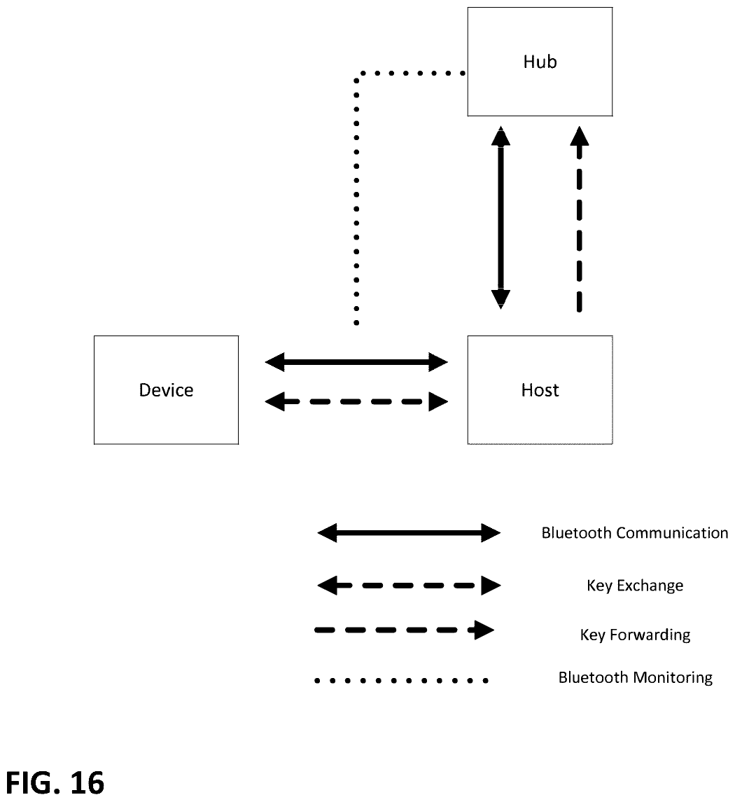

Encryption key passing may be used, for example, to covertly monitor a system. FIG. 16 illustrates an example of encryption key passing to enable discreet monitoring of devices. A host or central device (e.g., in Bluetooth connection to the device) may pass a copy of its encryption key to a surgical hub or surveilling device, for example, to enable the device to additionally monitor the communication between the device and host. The device may not have knowledge that the communication is being monitored, for example, because of the use of encryption key passing.

Identification systems may be used, for example, to covertly monitor a system. A system may be aware of adversaries within the operating room. The awareness may include awareness of devices that try to infiltrate surgical devices. A system may determine that a connected smart system used to acquire system information may be connected. A notification may be sent to notify to remove the competing device or the indicate to be on high alert for intrusion.

A system may be aware of friendly devices within an operating room. A visual identification marking may be used to detect friendly devices. Notification may be generated to HCPs to indicate compatible devices. A confirmation or rejection of compatible devices may enable the smart system to learn.

Instructions may be automatically displayed for friendly devices.

A system may use enabling technology. For example, a transponder may be used (e.g., transmitter and responder). The transponder may change responses based on received transmissions. A system may use automatic gain control, for example, to spoof real data by drowning it out with clutter and/or noise.

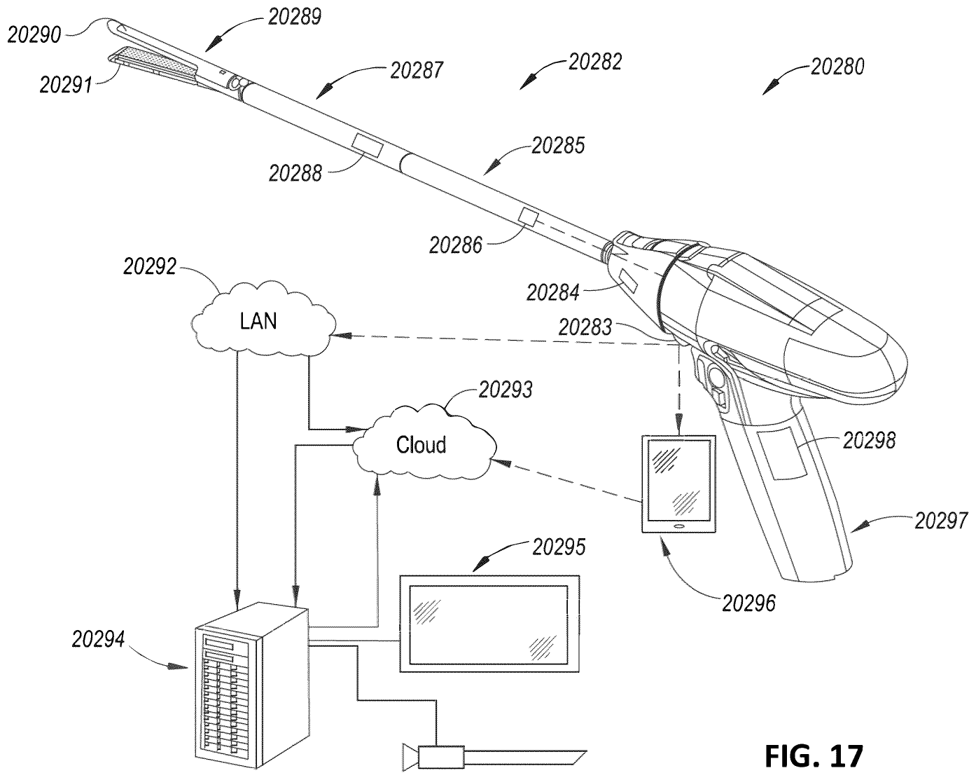

FIG. 17 illustrates an example surgical system 20280 that may include a surgical instrument 20282. The surgical instrument 20282 can be in communication with a console 20294 and/or a portable device 20296 through a local area network 20292 and/or a cloud network 20293 via a wired and/or wireless connection. The console 20294 and the portable device 20296 may be any suitable computing device. Surgical instrument 20282 may include a handle 20297, an adapter 20285, and a loading unit 20287. The adapter 20285 releasably couples to the handle 20297 and the loading unit 20287 releasably couples to the adapter 20285 such that the adapter 20285 transmits a force from a drive shaft to the loading unit 20287. The adapter 20285 or the loading unit 20287 may include a force gauge (not explicitly shown) disposed therein to measure a force exerted on the loading unit 20287. The loading unit 20287 may include an end effector 20289 having a first jaw 20291 and a second jaw 20290. The loading unit 20287 may be an in-situ loaded or multi-firing loading unit (MFLU) that allows a clinician to fire a plurality of fasteners multiple times without requiring the loading unit 20287 to be removed from a surgical site to reload the loading unit 20287.

The first and second jaws 20291, 20290 may be configured to clamp tissue therebetween, fire fasteners through the clamped tissue, and sever the clamped tissue. The first jaw 20291 may be configured to fire at least one fastener a plurality of times or may be configured to include a replaceable multi-fire fastener cartridge including a plurality of fasteners (e.g., staples, clips, etc.) that may be fired more than one time prior to being replaced. The second jaw 20290 may include an anvil that deforms or otherwise secures the fasteners, as the fasteners are ejected from the multi-fire fastener cartridge.

The handle 20297 may include a motor that is coupled to the drive shaft to affect rotation of the drive shaft. The handle 20297 may include a control interface to selectively activate the motor. The control interface may include buttons, switches, levers, sliders, touchscreens, and any other suitable input mechanisms or user interfaces, which can be engaged by a clinician to activate the motor.

The control interface of the handle 20297 may be in communication with a controller 20298 of the handle 20297 to selectively activate the motor to affect rotation of the drive shafts. The controller 20298 may be disposed within the handle 20297 and may be configured to receive input from the control interface and adapter data from the adapter 20285 or loading unit data from the loading unit 20287. The controller 20298 may analyze the input from the control interface and the data received from the adapter 20285 and/or loading unit 20287 to selectively activate the motor. The handle 20297 may also include a display that is viewable by a clinician during use of the handle 20297. The display may be configured to display portions of the adapter or loading unit data before, during, or after firing of the instrument 20282.

The adapter 20285 may include an adapter identification device 20284 disposed therein and the loading unit 20287 may include a loading unit identification device 20288 disposed therein. The adapter identification device 20284 may be in communication with the controller 20298, and the loading unit identification device 20288 may be in communication with the controller 20298. It will be appreciated that the loading unit identification device 20288 may be in communication with the adapter identification device 20284, which relays or passes communication from the loading unit identification device 20288 to the controller 20298.

The adapter 20285 may also include a plurality of sensors 20286 (one shown) disposed thereabout to detect various conditions of the adapter 20285 or of the environment (e.g., if the adapter 20285 is connected to a loading unit, if the adapter 20285 is connected to a handle, if the drive shafts are rotating, the torque of the drive shafts, the strain of the drive shafts, the temperature within the adapter 20285, a number of firings of the adapter 20285, a peak force of the adapter 20285 during firing, a total amount of force applied to the adapter 20285, a peak retraction force of the adapter 20285, a number of pauses of the adapter 20285 during firing, etc.). The plurality of sensors 20286 may provide an input to the adapter identification device 20284 in the form of data signals. The data signals of the plurality of sensors 20286 may be stored within or be used to update the adapter data stored within the adapter identification device 20284. The data signals of the plurality of sensors 20286 may be analog or digital. The plurality of sensors 20286 may include a force gauge to measure a force exerted on the loading unit 20287 during firing.

The handle 20297 and the adapter 20285 can be configured to interconnect the adapter identification device 20284 and the loading unit identification device 20288 with the controller 20298 via an electrical interface. The electrical interface may be a direct electrical interface (i.e., include electrical contacts that engage one another to transmit energy and signals therebetween). Additionally, or alternatively, the electrical interface may be a non-contact electrical interface to wirelessly transmit energy and signals therebetween (e.g., inductively transfer). It is also contemplated that the adapter identification device 20284 and the controller 20298 may be in wireless communication with one another via a wireless connection separate from the electrical interface.

The handle 20297 may include a transceiver 20283 that is configured to transmit instrument data from the controller 20298 to other components of the system 20280 (e.g., the LAN 20292, the cloud 20293, the console 20294, or the portable device 20296). The controller 20298 may also transmit instrument data and/or measurement data associated with one or more sensors 20286 to a surgical hub. The transceiver 20283 may receive data (e.g., cartridge data, loading unit data, adapter data, or other notifications) from the surgical hub 20270. The transceiver 20283 may receive data (e.g., cartridge data, loading unit data, or adapter data) from the other components of the system 20280. For example, the controller 20298 may transmit instrument data including a serial number of an attached adapter (e.g., adapter 20285) attached to the handle 20297, a serial number of a loading unit (e.g., loading unit 20287) attached to the adapter 20285, and a serial number of a multi-fire fastener cartridge loaded into the loading unit to the console 20294. Thereafter, the console 20294 may transmit data (e.g., cartridge data, loading unit data, or adapter data) associated with the attached cartridge, loading unit, and adapter, respectively, back to the controller 20298. The controller 20298 can display messages on the local instrument display or transmit the message, via transceiver 20283, to the console 20294 or the portable device 20296 to display the message on the display 20295 or portable device screen, respectively.

What is claimed is:

1. A method for operating a surgical system, comprising:
determining, using a first operating configuration, first data associated with the surgical system;
determining that the surgical system is being monitored based on the first data associated with the surgical system, wherein the first data is impacted based on the surgical system being monitored;
determining an impact associated with the surgical system being monitored;
determining an operating configuration to use based on the impact associated with the surgical system being monitored, wherein the determined operating configuration is the first operating configuration or a second operating configuration;
generating second data based on the determined operating configuration.

2. The method of claim 1, wherein the determination that the surgical system is being monitored based on the first data associated with the surgical system comprises one of a determination that a value associated with the first data is outside a threshold range associated with proper operation of the surgical system or a determination that the first data is inaccurate data.

3. The method of claim 1, wherein the determination of the impact associated with the surgical system being monitored is based on one or more of a type of data associated with the first data, a risk associated with operating using the impacted data, a type of monitoring that is being used to monitor the surgical system, or an importance associated with the first data, and wherein the type of monitoring is associated with whether the monitoring is observing data or editing the data.

4. The method of claim 1, wherein the determination of the operating configuration to be used based on the impact comprises a determination that the impact is greater than a threshold.

5. The method of claim 4, wherein the threshold is associated with a safe mode operation configuration, wherein based on the determination that the impact is greater than the threshold, the determined operating configuration is associated with at least one safe mode parameter for operating, and wherein the at least one safe mode parameter for operating is associated with a reduce functionality.

6. The method of claim 4, wherein the threshold is associated with a selective intervention operating configuration, wherein based on the determination that the impact is greater than the threshold, the determined operating configuration is associated with an operating configuration parameter, wherein the operating configuration parameter is associated with one or more of reducing collection of protected data or ensuring an integrity associated with generated data.

7. The method of claim 4, wherein based on the determination that the impact is greater than the threshold, the determined operating configuration uses a transform parameter, and wherein the second data is generated based on the transform parameter.

8. A method for operating a first surgical system, comprising:
obtaining, using a first operating configuration, first data associated with a second surgical system;
determining an impact associated with the second surgical system being monitored, wherein the impact is determined based on the first data;
determining an operating configuration to use based on the impact associated with the second surgical system being monitored, wherein the determined operating configuration is the first operating configuration or a second operating configuration; and
obtaining, using the determined operating configuration, second data associated with the second surgical system.

9. The method of claim 8, wherein the determination of the operating configuration to be used based on the impact comprises a determination that the impact is greater than a threshold.

10. The method of claim 8, wherein the impact associated with the second surgical system being monitored is associated with inaccurate data or degraded data

11. The method of claim 8, wherein the first operating configuration is associated with a first parameter and the second operating configuration is associated with a second parameter, and wherein the first parameter is associated with a first impedance, and wherein the second parameter is associated with a second impedance.

12. The method of claim 8, wherein the operating configuration is further determined based on one or more of a type associated with the data or a level associated with the data.

13. The method of claim 12, wherein the level associated with the data is a privacy level.

14. A surgical system, comprising:
a processor configured to:
determine, using a first operating configuration, first data associated with the surgical system;
determine that the surgical system is being monitored based on the first data associated with the surgical system, wherein the first data is impacted based on the surgical system being monitored;
determine an impact associated with the surgical system being monitored;
determine an operating configuration to use based on the impact associated with the surgical system being monitored, wherein the determined operating configuration is the first operating configuration or a second operating configuration;
generate second data based on the determined operating configuration.

15. The surgical system of claim 14, wherein the determination that the surgical system is being monitored based on the first data associated with the surgical system comprises one of a determination that a value associated with the first data is outside a threshold range associated with proper operation of the surgical system or a determination that the first data is inaccurate data.

16. The surgical system of claim 14, wherein the determination of the impact associated with the surgical system being monitored is based on one or more of a type of data associated with the first data, a risk associated with operating using the impacted data, a type of monitoring that is being used to monitor the surgical system, or an importance associated with the first data, and wherein the type of monitoring is associated with whether the monitoring is observing data or editing the data.

17. The surgical system of claim 14, wherein the determination of the operating configuration to be used based on the impact comprises a determination that the impact is greater than a threshold.

18. The surgical system of claim 17, wherein the threshold is associated with a safe mode operation configuration, wherein based on the determination that the impact is greater than the threshold, the determined operating configuration is associated with at least one safe mode parameter for operating, and wherein the at least one safe mode parameter for operating is associated with a reduce functionality.

19. The surgical system of claim 17, wherein the threshold is associated with a selective intervention operating configuration, wherein based on the determination that the impact is greater than the threshold, the determined operating configuration is associated with an operating configuration parameter, wherein the operating configuration parameter is associated with one or more of reducing collection of protected data or ensuring an integrity associated with generated data.

20. The surgical system of claim 17, wherein based on the determination that the impact is greater than the threshold, the determined operating configuration uses a transform parameter, and wherein the second data is generated based on the transform parameter.
